(19) United States
(12) Patent Application Publication
(10) Pub. No.: US 2022/0206019 A1
(43) Pub. Date: Jun. 30, 2022

(54) NEAR-INFRARED (NIR) QUALITY MONITORING METHOD USED IN COLUMN CHROMATOGRAPHY FOR EXTRACTING CONJUGATED ESTROGENS (CEs) FROM PREGNANT MARE URINE (PMU)

(71) Applicants: Xinjiang Medical University, Urumqi (CN); Xinjiang Nuziline Biopharmaceutical Co., Ltd, Yili Prefecture (CN)

(72) Inventors: Xiaoli Gao, Urumqi (CN); Xue Xiao, Urumqi (CN); Tuo Guo, Urumqi (CN); Jinfang Ma, Urumqi (CN); Jun Luo, Urumqi (CN); Zhiyong Xu, Urumqi (CN); Qunqun Huang, Urumqi (CN); Jiangbo Zeng, Urumqi (CN); Zhanying Chang, Urumqi (CN)

(21) Appl. No.: 17/510,667

(22) Filed: Oct. 26, 2021

(30) Foreign Application Priority Data
Dec. 25, 2020 (CN) 202011560084.6

(51) Int. Cl. G01N 33/74; G01N 21/359; G01N 21/03; G01N 21/3577

(52) U.S. Cl. CPC G01N 33/743; G01N 21/3577; G01N 21/0332; G01N 21/359

(57) ABSTRACT

A near-infrared (NIR) quality monitoring method used in column chromatography for extracting conjugated estrogens (CEs) from pregnant mare urine (PMU), that includes steps of: collecting an eluate obtained from column chromatography of a PMU stock solution as a to-be-tested sample; subjecting the to-be-tested sample to near-infrared spectroscopy (NIRS) to obtain raw spectral data, eliminating abnormal spectral values from the raw spectral data by a Mahalanobis distance method based on L1-PCA, and importing spectral data obtained after the abnormal spectral values are eliminated into a correction model to obtain a CE content in the to-be-tested sample; the correction model is a linear equation illustrating a relationship between true values and measured values, and the measured values refer to the NIR spectral data obtained after the abnormal spectral values are eliminated; and the CEs comprise one or more of sodium 17α-dihydroequilin sulfate, sodium equilin sulfate, and sodium estrone sulfate.

### CROSS REFERENCE TO RELATED APPLICATION(S)

This patent application claims the benefit and priority of Chinese Patent Application No. 202011560084.6, filed on Dec. 25, 2020, the disclosure of which is incorporated by reference herein in its entirety as part of the present application.

### TECHNICAL FIELD

The present disclosure relates to the technical field of quality monitoring, and in particular to a near-infrared (NIR) quality monitoring method used in column chromatography for extracting conjugated estrogens (CEs) from pregnant mare urine (PMU).

### BACKGROUND

It is well known that CEs are an effective drug for treating a menopausal syndrome. Natural CEs particularly have definite efficacy and reliable safety. Natural CEs can be used clinically not only to treat and prevent a menopausal syndrome occurring after female physiological or artificial menopause, but also to prevent and treat osteoporosis. CEs have been used and recognized by people for a long time.

Early-reported patents for CEs extraction methods include U.S. Pat. Nos. 2,429,398, 2,519,516, 2,696,265, 2,711,988, 2,834,712, etc., most of which provide an extraction method using an organic solvent. In the 1960s, methods such as activated carbon, ion-exchange resin, reverse phase silica gel, and the like were used to extract CEs from PMU. In current extraction methods, the CEs (substances with a steroidal structure) are separated and prepared mainly due to their hydrophobicity. Many invention patents related to the separation and preparation of CEs have been published at home and abroad, including reverse phase silica gel, macroporous resin with various functional groups, styrene-divinyl polymer non-polar resin, polyacrylate resin, strongly basic anion exchange resin with quaternary ammonium functional groups, and other adsorption resins.

Enrichment and extraction using macroporous adsorption resin are the key process steps for extracting CEs from PMU. In a conventional process monitoring method, samples are collected and sent to a laboratory to detect contents of sodium estrone sulfate, sodium equilin sulfate, etc., and it usually takes several hours or even a day to obtain results, which lags behind a column chromatographic process and cannot realize the process control of a column chromatographic process.

In the field of near-infrared spectroscopy (NIRS) analysis, the Mahalanobis distance method is often used to eliminate abnormal spectra, but the Mahalanobis distance method requires the total number of samples to be greater than the dimension of samples, resulting in cumbersome processing.

### SUMMARY

The present disclosure is intended to provide an NIR quality monitoring method used in column chromatography for extracting CEs from PMU. The method provided in the present disclosure can quickly evaluate the quality of a PMU eluate obtained from column chromatography to extract CEs from PMU. Compared with a conventional method of sampling and conducting liquid chromatography (LC) detection, the method of the present disclosure is more time-saving and pollution-free, and saves a lot of manpower and material resources. The present disclosure uses a Mahalanobis distance method based on L1-PCA to eliminate abnormal spectral values, which can significantly improve the accuracy of detection results.

To achieve the above purpose, the present disclosure provides the following technical solutions.

An NIR quality monitoring method used in column chromatography for extracting CEs from PMU includes the following steps:

collecting an eluate obtained from column chromatography of a PMU stock solution as a to-be-tested sample;

subjecting the to-be-tested sample to near-infrared spectroscopy (NIRS) to obtain raw spectral data, eliminating abnormal spectral values from the raw spectral data by a Mahalanobis distance method based on L1-PCA, and importing spectral data obtained after the abnormal spectral values are eliminated into a correction model to obtain a CE content in the to-be-tested sample;

where, the correction model is a linear equation illustrating a relationship between true values and measured values, and the measured values refer to the NIR spectral data obtained after the abnormal spectral values are eliminated; and

the CEs include one or more of sodium 17α-dihydroequilin sulfate, sodium equilin sulfate, and sodium estrone sulfate.

Preferably, a method for building the correction model may include the following steps:

(1) subjecting the PMU stock solution to column chromatography to obtain a PMU eluate sample;

(2) subjecting the PMU eluate sample to liquid chromatography (LC) detection to obtain an actual CE content value in the PMU eluate sample;

(3) subjecting the PMU eluate sample in step (1) to NIRS to obtain raw sample spectral data, eliminating abnormal sample spectral values by the Mahalanobis distance method based on L1-PCA, and acquiring spectral data of the PMU eluate sample; and

(4) pre-processing the spectral data acquired in step (3), and subjecting pre-processed spectral data to band selection to obtain characteristic bands; and with partial least squares (PLS), subjecting spectral data of a characteristic band and a corresponding actual CE content value in the PMU eluate sample to regression fit to build a correction model;

where, steps (2) and (3) can be executed in any order.

Preferably, correction models for different CEs may be as follows:

a correction model for sodium 17α-dihydroequilin sulfate: y=0.9173x+0.0128;

a correction model for sodium equilin sulfate: y=0.9079x+0.0258;

a correction model for sodium estrone sulfate: y=0.9151x+0.0396; and

a correction model for sodium equilin sulfate+sodium estrone sulfate: y=0.9148x++0.0636; and

in the above correction models, x represents a true value and y represents a predicted value.

Preferably, when a total content of CEs in the to-be-tested sample is greater than 0.001 mg/mL, it may be determined as a starting point of the column chromatographic elution for PMU; and

when a total content of CEs in the to-be-tested sample is less than 0.001 mg/mL, it may be determined as an end point of the column chromatographic elution for PMU.

Preferably, the eliminating abnormal spectral values by the Mahalanobis distance method based on L1-PCA may include:

building a spectral matrix from the raw spectral data;

according to a calculation formula shown in formula I, using an L1-PCA algorithm to solve the spectral matrix to obtain spectral principal components;

building a covariance matrix from the principal components according to a calculation formula shown in formula II;

calculating a Mahalanobis distance from the covariance matrix according to a calculation formula shown in formula III; and

setting a threshold and eliminating abnormal spectral values; where,

E₂(U,V)=min∥X′−UV∥_{L}_{₁}, formula I;

in formula I, X′ is an n×m spectral sample matrix, with n as the number of samples and m as the number of data points acquired for each spectrum; U is a projection matrix; V is a coefficient matrix; and L₁ is matrix norm 1;

S=T′T/n, formula II;

in formula II, T′ is the transposition of T, n is the number of samples, and a calculation method of T includes: after a signal subspace P of spectral data is obtained, calculating a mean spectral vector μ according to the P, and subtracting the mean spectral vector μ from each sample of the P matrix;

D=\sqrt{(P−μ)^{T}S⁻¹(P−μ)}, formula III;

in formula III, P is the signal subspace of spectral data; μ is the mean spectral vector; and S is a covariance matrix of the sample signal subspace built from T;

the threshold is 2 to 3.

Preferably, parameters for the LC detection in step (2) may include:

chromatographic column: C18 chromatographic column;

chromatographic column specification: 250 mm×4.6 mm, 5 μm, 100 A;

mobile phase: phase A and phase B, where, the phase A is a mixed solution of a monosodium phosphate (MSP) aqueous solution, acetonitrile, and methanol in a volume ratio of 17:2:1, and the MSP aqueous solution has a concentration of 20 mmol/L and a pH of 3.5; and the phase B is a mixed solution of a disodium phosphate (DSP) aqueous solution and acetonitrile in a volume ratio of 3:7, and the DSP aqueous solution has a concentration of 10 mmol/L and a pH of 3.5;

elution procedure in the mobile phase: 0 min to 18 min, a volume fraction of phase A: reducing from 70% to 67%; 18 min to 23 min, a volume fraction of phase A: reducing from 67% to 20%; 23 min to 28 min, a volume fraction of phase A: increasing from 20% to 70%; and 28 min to 35 min, a volume fraction of phase A: stabilizing at 70%;

flow rate: 1.0 mL/min;

column temperature: 40° C.;

detection wavelength: 205 nm; and

injection volume: 1 μL.

Preferably, the NIRS may be conducted under the following conditions:

on-line or off-line detection; background: air; transmission measurement mode; wavelength detection range: 10,000 cm⁻¹ to 4,000 cm⁻¹; number of scans: 32; resolution: 8 cm⁻¹; optical path length (OPL): 2 mm; 3 to 5 repetitive scans for each to-be-tested sample; and raw spectral data: average value;

or, based on the principle of raster scanning spectroscopy, light source: tungsten halogen lamp; spectral range: 1,000 nm to 1,800 nm; detector: InGaAs detector; resolution: 8 cm⁻¹; number of scans: 32; and OPL: 1 mm.

Preferably, a method for the pre-processing in step (4) may include: one of convolution-based smoothing, first order convolution-based derivation, second order convolution-based derivation, multiplicative scatter correction (MSC), standard normal variant (SNV) transformation, and normalization, or a combination of two or more thereof.

Preferably, a method of the band selection in step (4) may include full wavelength, correlation-coefficient method for wavelength interval selection, correlated component method for wavelength interval selection, iterative optimization wavelength selection method 1, or iterative optimization wavelength selection method 2.

The present disclosure provides an NIR quality monitoring method used in column chromatography for extracting CEs from PMU. The present disclosure builds a correction model, which is a linear equation illustrating a relationship between true values and measured values, and the measured values refer to the NIR spectral data obtained after the abnormal spectral values are eliminated. The present disclosure uses the Mahalanobis distance method based on L1-PCA to eliminate abnormal spectral values, which can significantly improve the accuracy of detection results. The present disclosure adopts the Mahalanobis distance method based on L1-PCA to eliminate overlapping information parts in a large amount of coexist information through data dimension reduction, which is more convenient for the processing of a small number of samples, suppresses a heavy-tailed noise, and improves the identifiability of a signal. In addition, when the number of extracted features is small, the Mahalanobis distance method based on L1-PCA is more suitable for the elimination of abnormal spectra.

The method of the present disclosure can quickly evaluate the quality of a PMU eluate obtained from column chromatography to extract CEs from PMU. Compared with a conventional method of sampling and conducting HPLC detection, the method of the present disclosure is more time-saving and pollution-free, and saves a lot of manpower and material resources. From another perspective, for the quality monitoring of column chromatography for extracting CEs from PMU, on the one hand, a starting point and end point can be determined for the column chromatographic elution, on the other hand, main index components for quality control (sodium 17α-dihydroequilin sulfate, sodium equilin sulfate, sodium estrone sulfate, and sodium equilin sulfate+sodium estrone sulfate) can be monitored during the column chromatographic process.

### BRIEF DESCRIPTION OF THE DRAWINGS

FIG. 1 is a data diagram illustrating sodium 17α-dihydroequilin sulfate contents detected by LC;

FIG. 2 is a data diagram illustrating sodium equilin sulfate contents detected by LC;

FIG. 3 is a data diagram illustrating sodium estrone sulfate contents detected by LC;

FIG. 4 is a data diagram illustrating sodium equilin sulfate+sodium estrone sulfate contents detected by LC;

FIG. 5 shows NIR spectra of CEs;

FIG. 6 shows abnormal spectrum calculation results of the Mahalanobis distance method based on L1-PCA;

FIG. 7 shows abnormal spectrum calculation results of the Mahalanobis distance method;

FIG. 8 is a content trend graph of the modeling sample set of sodium 17α-dihydroequilin sulfate;

FIG. 9 is a predicted trend graph of sodium 17α-dihydroequilin sulfate in samples of batch 20181211-2;

FIG. 10 is a predicted trend graph obtained after the abnormal samples in FIG. 9 are eliminated;

FIG. 11 is a content trend graph of the modeling sample set of sodium equilin sulfate;

FIG. 12 is a predicted trend graph of sodium equilin sulfate in samples of batch 20181211-2;

FIG. 13 is a predicted trend graph obtained after the abnormal samples in FIG. 12 are eliminated;

FIG. 14 is a content trend graph of the modeling sample set of sodium estrone sulfate;

FIG. 15 is a predicted trend graph of sodium estrone sulfate in samples of batch 20181211-2;

FIG. 16 is a predicted trend graph obtained after the abnormal samples in FIG. 15 are eliminated;

FIG. 17 is a content trend graph of the modeling sample set of sodium equilin sulfate+sodium estrone sulfate;

FIG. 18 is a predicted trend graph of sodium equilin sulfate+sodium estrone sulfate in samples of batch 20181211-2; and

FIG. 19 is a predicted trend graph obtained after the abnormal samples in FIG. 17 are eliminated.

### DETAILED DESCRIPTION

The present disclosure provides an NIR quality monitoring method used in column chromatography for extracting CEs from PMU, including the following steps:

collecting an eluate obtained from column chromatography of a PMU stock solution as a to-be-tested sample;

subjecting the to-be-tested sample to NIRS to obtain raw spectral data, eliminating abnormal spectral values from the raw spectral data by a Mahalanobis distance method based on L1-PCA, and importing spectral data obtained after the abnormal spectral values are eliminated into a correction model to obtain a CE content in the to-be-tested sample;

where, the correction model is a linear equation illustrating a relationship between true values and measured values, and the measured values refer to the NIR spectral data obtained after the abnormal spectral values are eliminated; and

the CEs include one or more of sodium 17α-dihydroequilin sulfate, sodium equilin sulfate, and sodium estrone sulfate.

In the present disclosure, an eluate obtained from column chromatography of a PMU stock solution is collected as a to-be-tested sample. In the present disclosure, a stationary phase for the column chromatography may preferably be a macroporous resin, and a mobile phase may preferably be ethanol. The present disclosure has no special requirements for specific process parameters of the column chromatography, and a process well known to those skilled in the art may be adopted.

In the present disclosure, after a to-be-tested sample is obtained, the to-be-tested sample is subjected to NIRS to obtain raw spectral data, abnormal spectral values are eliminated from the raw spectral data by a Mahalanobis distance method based on L1-PCA, and spectral data obtained after the abnormal spectral values are eliminated are imported into a correction model to obtain a CE content in the to-be-tested sample. The correction model is a linear equation illustrating a relationship between true values and measured values, and the measured values refer to the NIR spectral data obtained after the abnormal spectral values are eliminated; and the CEs include one or more of sodium 17α-dihydroequilin sulfate, sodium equilin sulfate, and sodium estrone sulfate.

In the present disclosure, the NIRS may preferably be conducted under the following conditions:

on-line or off-line detection; background: air; transmission measurement mode; wavelength detection range: 10,000 cm⁻¹ to 4,000 cm⁻¹; number of scans: 32; resolution: 8 cm⁻¹; optical path length (OPL): 2 mm; 3 to 5 repetitive scans for each test solution; and spectral data: average value;

or, based on the principle of raster scanning spectroscopy, light source: tungsten halogen lamp; spectral range: 1,000 nm to 1,800 nm; detector: InGaAs detector; resolution: 8 cm⁻¹; number of scans: 32; and OPL: 1 mm.

In the present disclosure, each scan takes 3 s to 5 s on average.

In the present disclosure, the eliminating abnormal spectral values by a Mahalanobis distance method based on L1-PCA may preferably include:

building a spectral matrix from the raw spectral data;

according to a calculation formula shown in formula I, using an L1-PCA algorithm to solve the spectral matrix to obtain spectral principal components;

building a covariance matrix from the principal components according to a calculation formula shown in formula II;

calculating a Mahalanobis distance from the covariance matrix according to a calculation formula shown in formula III; and

setting a threshold and eliminating abnormal spectral values; where,

E₂(U,V)=min∥X′−UV∥_{L}_{₁}, formula I;

in formula I, X′ is an n×m spectral sample matrix, with n as the number of samples and m as the number of data points acquired for each spectrum; U is a projection matrix; V is a coefficient matrix; and L₁ is matrix norm 1;

S=T′T/n, formula II;
in formula II, T′ is the transposition of T, n is the number of samples, and a calculation method of T includes: after a signal subspace P of spectral data is obtained, calculating a mean spectral vector μ according to the P, and subtracting the mean spectral vector μ from each sample of the P matrix;

D=\sqrt{(P−μ)^{T}S⁻¹(P−μ)}, formula III;

in formula III, P is the signal subspace of spectral data; μ is the mean spectral vector; and S is a covariance matrix of the sample signal subspace built from T; and

the threshold is 2 to 3.

In a specific example of the present disclosure, the using an L1-PCA algorithm to solve the spectral matrix to obtain spectral principal components refers to solving an optimization problem. When an optimization problem of formula I is solved, as an objective function constituted of the L₁ norm is not a convex function, it is not directly solved by a convex optimization algorithm. U and V are alternately assumed be known, the cost function becomes a convex function, and then the convex optimization algorithm is used to solve the problem.

In the present disclosure, during the building a covariance matrix from the principal components according to a calculation formula shown in formula II, corresponding characteristic values of selected principal components account for more than 95% of a sum of all characteristic values.

In a specific example of the present disclosure, a calculated Mahalanobis distance is the Mahalanobis distance after L1 norm-constrained principal component analysis (PCA).

In a specific example of the present disclosure, the threshold is 2.5. In the present disclosure, abnormal sample spectral values are eliminated according to a threshold range.

In the present disclosure, a method for building the correction model may preferably include the following steps:

(1) subjecting the PMU stock solution to column chromatography to obtain a PMU eluate sample;

(2) subjecting the PMU eluate sample to LC detection to obtain an actual CE content value in the PMU eluate sample;

(3) subjecting the PMU eluate sample in step (1) to NIRS to obtain raw sample spectral data, eliminating abnormal sample spectral values by the Mahalanobis distance method based on L1-PCA, and acquiring spectral data of the PMU eluate sample; and

(4) pre-processing the spectral data acquired in step (3), and subjecting pre-processed spectral data to band selection to obtain characteristic bands; and with partial least squares (PLS), subjecting spectral data of a characteristic band and a corresponding actual CE content value in the PMU eluate sample to regression fit to build a correction model;

where, steps (2) and (3) can be executed in any order.

In the present disclosure, a column chromatography process for building the correction model is the same as that for collecting the to-be-tested sample, which will not be repeated here.

In the present disclosure, parameters for the LC detection may preferably include:

chromatographic column: C18 chromatographic column;

chromatographic column specification: 250 mm×4.6 mm, 5 μm, 100 A;

mobile phase: phase A and phase B, where, the phase A is a mixed solution of a monosodium phosphate (MSP) aqueous solution, acetonitrile, and methanol in a volume ratio of 17:2:1, and the MSP aqueous solution has a concentration of 20 mmol/L and a pH of 3.5; and the phase B is a mixed solution of a disodium phosphate (DSP) aqueous solution and acetonitrile in a volume ratio of 3:7, and the DSP aqueous solution has a concentration of 10 mmol/L and a pH of 3.5;

elution procedure in the mobile phase: 0 min to 18 min, a volume fraction of phase A: reducing from 70% to 67%; 18 min to 23 min, a volume fraction of phase A: reducing from 67% to 20%; 23 min to 28 min, a volume fraction of phase A: increasing from 20% to 70%; and 28 min to 35 min, a volume fraction of phase A: stabilizing at 70%;

flow rate: 1.0 mL/min;

column temperature: 40° C.;

detection wavelength: 205 nm; and

injection volume: 1 μL.

Peaks for different CEs appear at different retention times under the same chromatographic conditions.

In the present disclosure, after the PMU eluate sample is subjected to LC detection, abnormal data values may preferably be eliminated to obtain an actual CE content value in the PMU eluate sample. The present disclosure has no special requirements for a method to eliminate abnormal data values, and a method well known to those skilled in the art may be adopted. In a specific example of the present disclosure, abnormal data obtained by the LC detection can be visually observed and thus can be directly eliminated.

In the present disclosure, the PMU eluate sample is subjected to NIRS to obtain raw sample spectral data, abnormal sample spectral values are eliminated by the Mahalanobis distance method based on L1-PCA, and spectral data of the PMU eluate sample are acquired. In the present disclosure, parameters for the NIRS and a method for eliminating abnormal sample spectral values by the Mahalanobis distance method based on L1-PCA are the same as that used in the detection of the to-be-tested sample described above, which will not be repeated here.

In the present disclosure, after spectral data of the PMU eluate sample are obtained, the spectral data acquired are pre-processed, and pre-processed spectral data are subjected to band selection to obtain characteristic bands; and with PLS, spectral data of a characteristic band and a corresponding actual CE content value in the PMU eluate sample are subjected to regression fit to build a correction model.

In the present disclosure, a method for the pre-processing may preferably include: one of convolution-based smoothing, first order convolution-based derivation, second order convolution-based derivation, multiplicative scatter correction (MSC), SNV transformation, and normalization, or a combination of two or more thereof, and more preferably convolution-based smoothing.

In the present disclosure, a method for the band selection may preferably include full wavelength, correlation-coefficient method for wavelength interval selection, correlated component method for wavelength interval selection, iterative optimization wavelength selection method 1, or iterative optimization wavelength selection method 2, and more preferably iterative optimization wavelength selection method 1. In the present disclosure, the iterative optimization wavelength selection method 1 includes: conducting full permutation and combination on N wavelength intervals, using each combination for modeling, and selecting the one with the smallest SECV as the optimal model for this optimization; and the iterative optimization wavelength selection method 2 includes: selecting M intervals from N wavelength intervals to form a spectrum for modeling, namely, selecting M from N, subjecting all possible combinations to modeling, and selecting the one with the smallest SECV as the optimal model for this optimization, where, N is 10 and M is 1, 2, or 3.

In the present disclosure, the CEs may include one or more of sodium 17α-dihydroequilin sulfate, sodium equilin sulfate, and sodium estrone sulfate, and preferably include one or more of 17α-dihydroequilin sulfate, sodium equilin sulfate, sodium estrone sulfate, and sodium equilin sulfate+sodium estrone sulfate, where, the sodium equilin sulfate+sodium estrone sulfate means that a sum of contents of the two is used as an index for building a correction model.

In the present disclosure, correction models for different CEs may preferably be as follows:

a correction model for sodium 17α-dihydroequilin sulfate: y=0.9173x+0.0128;

a correction model for sodium equilin sulfate: y=0.9079x+0.0258;

a correction model for sodium estrone sulfate: y=0.9151x+0.0396; and

a correction model for sodium equilin sulfate+sodium estrone sulfate: y=0.9148x++0.0636.

In the above correction models, x represents a true value and y represents a predicted value.

In a specific example of the present disclosure, the correction models for different CEs are shown in Table 1:

TABLE 1
Correction models for different CEs

| | | | | | | Predicted | | |
|---|---|---|---|---|---|---|---|---|
| | Pre-processing | | Number of | | | value-true | | |
| | method for | Band | principal | | | value fitting | Determination | |
| Component | spectrum | selection | factors | SECV | SEC | equation | coefficient | Offset |
| Sodium | Convolution- | Iterative | 10 | 0.1237 | 0.0973 | y = 0.9173 | 91.73 | 0.0012 |
| 17 α- | based | optimization | | | | x + 0.0128 | | |
| dihydroequilin | smoothing | wavelength | | | | | | |
| sulfate | | selection | | | | | | |
| | | method 1 | | | | | | |
| Sodium equilin | Convolution- | Iterative | 10 | 0.2340 | 0.1722 | y = 0.9079 | 90.79 | 0.0014 |
| sulfate | based | optimization | | | | x + 0.0258 | | |
| | smoothing | wavelength | | | | | | |
| | | selection | | | | | | |
| | | method 1 | | | | | | |
| Sodium estrone | Convolution- | Iterative | 10 | 0.3925 | 0.2888 | y = 0.9151 | 91.51 | 0.0023 |
| sulfate | based | optimization | | | | x + 0.0396 | | |
| | smoothing | wavelength | | | | | | |
| | | selection | | | | | | |
| | | method 1 | | | | | | |
| Sodium equilin | Convolution- | Iterative | 10 | 0.6192 | 0.4543 | y = 0.9148 | 91.48 | 0.0037 |
| sulfate + sodium | based | optimization | | | | x + 0.0636 | | |
| estrone sulfate | smoothing | wavelength | | | | | | |
| | | selection | | | | | | |
| | | method 1 | | | | | | |

In the predicted value-true value fitting equation in Table 1, x represents a true value and y represents a predicted value.

In a specific example of the present disclosure, spectral data obtained by subjecting a PMU eluate during a column chromatographic process to NIRS are imported into a correction model as a predicted value to obtain an actual CE content value in the PMU eluate during the column chromatographic process, thus achieving the quality monitoring of the PMU column chromatography process.

In the present disclosure, when a content of CEs in the PMU eluate is greater than 0.001 mg/mL, it is determined as a starting point of the column chromatographic elution for PMU; and when a content of CEs in the PMU eluate is less than 0.001 mg/mL, it is determined as an end point of the column chromatographic elution for PMU.

With the method provided in the present disclosure, a starting point and an end point of the column chromatographic elution can be determined in time, and thus the column chromatographic process can be accurately controlled.

The technical solutions of the present disclosure will be clearly and completely described below with reference to examples of the present disclosure. Apparently, the described examples are merely some rather than all of the examples of the present disclosure. All other examples obtained by a person of ordinary skill in the art based on the examples of the present disclosure without creative efforts shall fall within the protection scope of the present disclosure.

Experimental instruments used in the examples:

high-performance liquid chromatography (HPLC) instrument, waters 2996, America (including gradient pump G1311A, autosampler G1329A, column constant temperature system G1316A, diode-array detector (DAD) DAD-G1315B, chromatographic workstation).

Experimental Reagents Used:

phosphoric acid (analytical grade, Guangzhou Chemical Reagent Factory), methanol and acetonitrile (chromatographic grade, Merck, Germany), water (Watsons Co., Ltd.).

Experimental Materials:

PMU eluate samples (180, provided by Xinjiang Xinziyuan Biopharmaceutical Co., Ltd.), and a mixed standard of sodium 17α-dihydroequilin sulfate, sodium equilin sulfate, and sodium estrone sulfate (provided by Xinjiang Xinziyuan Biopharmaceutical Co., Ltd.)

### Example 1

(1) PMU stock solutions were subjected to elution with macroporous resin to obtain PMU eluate samples in different batches;

(2) the PMU eluate sample was subjected to LC detection to obtain an actual CE content value in the PMU eluate sample; and parameters for the LC detection were as follows:

chromatographic column: Sharpsil-UC18;

chromatographic column specification: 250 mm×4.6 mm, 5 μm, 100 A;

mobile phase: phase A and phase B, where, the phase A was a mixed solution of an MSP aqueous solution, acetonitrile, and methanol in a volume ratio of 17:2:1, and the MSP aqueous solution had a concentration of 20 mmol/L and a pH of 3.5; and the phase B was a mixed solution of a DSP aqueous solution and acetonitrile in a volume ratio of 3:7, and the DSP aqueous solution had a concentration of 10 mmol/L and a pH of 3.5;

elution procedure in the mobile phase: 0 min to 18 min, a volume fraction of phase A: reducing from 70% to 67%; 18 min to 23 min, a volume fraction of phase A: reducing from 67% to 20%; 23 min to 28 min, a volume fraction of phase A: increasing from 20% to 70%; and 28 min to 35 min, a volume fraction of phase A: stabilizing at 70%;

flow rate: 1.0 mL/min;

column temperature: 40° C.;

detection wavelength: 205 nm; and

injection volume: 1 μL.

Peaks for different CEs appeared at different retention times under the same chromatographic conditions.

Obtained actual CE content values in the PMU eluate samples were shown in Table 2 below.

TABLE 2
Results of content values of quality
control index components among CEs

| | | Content (mg/mL) | | | |
|---|---|---|---|---|---|
| | | | | | Sodium |
| | | Sodium | | | equilin |
| | | 17α- | | | sulfate + |
| | | dihydro- | Sodium | Sodium | sodium |
| Serial | | equilin | equilin | estrone | estrone |
| No. | Sample No. | sulfate | sulfate | sulfate | sulfate |
| 1 | MTC20181209-1-1 | 0.0046 | 0.1389 | 0.0130 | 0.1519 |
| 2 | MTC20181209-1-2 | 0.9462 | 1.394 | 2.8109 | 4.2055 |
| 3 | MTC20181209-1-3 | 1.3380 | 2.2249 | 4.0680 | 6.2929 |
| 4 | MTC20181209-1-4 | 0.9524 | 1.7819 | 3.2885 | 5.0704 |
| 5 | MTC20181209-1-5 | 0.5195 | 1.1234 | 2.0847 | 3.2081 |
| 6 | MTC20181209-1-6 | 0.2888 | 0.6785 | 1.1426 | 1.8211 |
| 7 | MTC20181209-1-7 | 0.1691 | 0.4211 | 0.6913 | 1.1124 |
| 8 | MTC20181209-1-8 | 0.1122 | 0.2957 | 0.4864 | 0.7821 |
| 9 | MTC20181209-1-9 | 0.0513 | 0.1531 | 0.2538 | 0.4069 |
| 10 | MTC20181209-1-10 | 0.1733 | 0.4305 | 0.7255 | 1.1561 |
| 11 | MTC20181209-1-11 | 0.0152 | 0.0473 | 0.0840 | 0.1313 |
| 12 | MTC20181209-1-12 | 0.0098 | 0.0295 | 0.0514 | 0.0809 |
| 13 | MTC20181209-1-13 | 0.0054 | 0.0180 | 0.0318 | 0.0498 |
| 14 | MTC20181209-1-14 | 0.0039 | 0.0136 | 0.0239 | 0.0375 |
| 15 | MTC20181209-1-15 | 0.0028 | 0.0100 | 0.0160 | 0.0261 |
| 16 | MTC20181209-1-16 | 0.0027 | 0.0080 | 0.0136 | 0.0216 |
| 17 | MTC20181209-1-17 | 0.0025 | 0.0081 | 0.0109 | 0.0190 |
| 18 | MTC20181209-1-18 | 0.0017 | 0.0043 | 0.0076 | 0.0119 |
| 19 | MTC20181209-1-19 | 0.0018 | 0.0040 | 0.0067 | 0.0107 |
| 20 | MTC20181209-1-20 | 0.0016 | 0.0031 | 0.0050 | 0.0081 |
| 21 | MTC20181209-1-21 | 0.0011 | 0.0023 | 0.0040 | 0.0063 |
| 22 | MTC20181209-1-22 | 0.0011 | 0.0018 | 0.0032 | 0.0050 |
| 23 | MTC20181209-1-23 | 0.0007 | 0.0011 | 0.0022 | 0.0033 |
| 24 | MTC20181209-1-24 | 0.0009 | 0.0012 | 0.0020 | 0.0032 |
| 25 | MTC20181209-1-25 | 0.0009 | 0.0000 | 0.0008 | 0.0008 |
| 26 | MTC20181209-1-26 | 0.0006 | 0.0000 | 0.0006 | 0.0006 |
| 27 | MTC20181209-1-27 | 0.0005 | 0.0000 | 0.0008 | 0.0008 |
| 28 | MTC20181209-1-28 | 0.0005 | 0.0000 | 0.0012 | 0.0012 |
| 29 | MTC20181209-1-29 | 0.0009 | 0.0000 | 0.0013 | 0.0013 |
| 30 | MTC20181209-1-30 | 0.0009 | 0.0000 | 0.0011 | 0.0011 |
| 31 | MTC20181209-2-1 | 0.2301 | 0.3439 | 0.6093 | 0.9532 |
| 32 | MTC20181209-2-2 | 0.9438 | 1.6745 | 2.8232 | 4.4978 |
| 33 | MTC20181209-2-3 | 0.6221 | 1.1352 | 1.7742 | 2.9094 |
| 34 | MTC20181209-2-4 | 0.3657 | 0.7263 | 1.1068 | 1.8331 |
| 35 | MTC20181209-2-5 | 0.1479 | 0.3022 | 0.4676 | 0.7698 |
| 36 | MTC20181209-2-6 | 0.2174 | 0.4184 | 0.6455 | 1.0639 |
| 37 | MTC20181209-2-7 | 0.0803 | 0.1774 | 0.2783 | 0.4557 |
| 38 | MTC20181209-2-8 | 0.0553 | 0.1254 | 0.1975 | 0.3229 |
| 39 | MTC20181209-2-9 | 0.0368 | 0.0842 | 0.1336 | 0.2178 |
| 40 | MTC20181209-2-10 | 0.0257 | 0.0606 | 0.0955 | 0.1561 |
| 41 | MTC20181209-2-11 | 0.0147 | 0.0371 | 0.0581 | 0.0952 |
| 42 | MTC20181209-2-12 | 0.0128 | 0.0301 | 0.0472 | 0.0773 |
| 43 | MTC20181209-2-13 | 0.0095 | 0.0229 | 0.0352 | 0.0581 |
| 44 | MTC20181209-2-14 | 0.0072 | 0.0184 | 0.0286 | 0.0470 |
| 45 | MTC20181209-2-15 | 0.0058 | 0.0139 | 0.0223 | 0.0361 |
| 46 | MTC20181209-2-16 | 0.0025 | 0.0068 | 0.0109 | 0.0178 |
| 47 | MTC20181209-2-17 | 0.0025 | 0.0052 | 0.0088 | 0.0140 |
| 48 | MTC20181209-2-18 | 0.0029 | 0.0068 | 0.0108 | 0.0176 |
| 49 | MTC20181209-2-19 | 0.0009 | 0.0019 | 0.0038 | 0.0057 |
| 50 | MTC20181209-2-20 | 0.0009 | 0.0015 | 0.0026 | 0.0040 |
| 51 | MTC20181209-2-21 | 0.0010 | 0.0014 | 0.0028 | 0.0043 |
| 52 | MTC20181209-2-22 | 0.0006 | 0.0015 | 0.0023 | 0.0039 |
| 53 | MTC20181209-2-23 | 0.0009 | 0.0016 | 0.0031 | 0.0048 |
| 54 | MTC20181209-2-24 | 0.0068 | 0.0129 | 0.0205 | 0.0335 |
| 55 | MTC20181209-2-25 | 0.0018 | 0.0034 | 0.0065 | 0.0099 |
| 56 | MTC20181209-2-26 | 0.0004 | 0.0014 | 0.0021 | 0.0035 |
| 57 | MTC20181209-2-27 | 0.0011 | 0.0015 | 0.0026 | 0.0041 |
| 58 | MTC20181209-2-28 | 0.0033 | 0.0068 | 0.0114 | 0.0182 |
| 59 | MTC20181209-2-29 | 0.0023 | 0.0050 | 0.0087 | 0.0136 |
| 60 | MTC20181209-2-30 | 0.0092 | 0.0172 | 0.0292 | 0.0465 |
| 61 | MTC20181210-1-1 | 0.0061 | 0.4930 | 0.0211 | 0.5141 |
| 62 | MTC20181210-1-2 | 0.5423 | 0.8609 | 1.5691 | 2.4300 |
| 63 | MTC20181210-1-3 | 1.0400 | 1.8438 | 3.4601 | 5.3039 |
| 64 | MTC20181210-1-4 | 0.7997 | 1.4445 | 2.5676 | 4.0121 |
| 65 | MTC20181210-1-5 | 0.4399 | 0.7856 | 1.3033 | 2.0889 |
| 66 | MTC20181210-1-6 | 0.2678 | 0.4493 | 0.7181 | 1.1674 |
| 67 | MTC20181210-1-7 | 0.1685 | 0.2693 | 0.4345 | 0.7038 |
| 68 | MTC20181210-1-8 | 0.1207 | 0.1944 | 0.3096 | 0.5040 |
| 69 | MTC20181210-1-9 | 0.0882 | 0.1422 | 0.2325 | 0.3747 |
| 70 | MTC20181210-1-10 | 0.0680 | 0.1133 | 0.1874 | 0.3007 |
| 71 | MTC20181210-1-11 | 0.0448 | 0.0784 | 0.1296 | 0.2080 |
| 72 | MTC20181210-1-12 | 0.0309 | 0.0610 | 0.0949 | 0.1559 |
| 73 | MTC20181210-1-13 | 0.0343 | 0.0690 | 0.1047 | 0.1737 |
| 74 | MTC20181210-1-14 | 0.0245 | 0.0435 | 0.0665 | 0.1100 |
| 75 | MTC20181210-1-15 | 0.0127 | 0.0282 | 0.0443 | 0.0726 |
| 76 | MTC20181210-1-16 | 0.0063 | 0.0158 | 0.0236 | 0.0394 |
| 77 | MTC20181210-1-17 | 0.0041 | 0.0119 | 0.0178 | 0.0297 |
| 78 | MTC20181210-1-18 | 0.0025 | 0.0083 | 0.0118 | 0.0201 |
| 79 | MTC20181210-1-19 | 0.0027 | 0.0046 | 0.0061 | 0.0107 |
| 80 | MTC20181210-1-20 | 0.0015 | 0.0035 | 0.0047 | 0.0082 |
| 81 | MTC20181210-1-21 | 0.0014 | 0.0023 | 0.0025 | 0.0048 |
| 82 | MTC20181210-1-22 | 0.0008 | 0.0016 | 0.0020 | 0.0036 |
| 83 | MTC20181210-1-23 | 0.0011 | 0.0023 | 0.0020 | 0.0044 |
| 84 | MTC20181210-1-24 | 0.0001 | 0.0000 | 0.0000 | 0.0000 |
| 85 | MTC20181210-1-25 | 0.0001 | 0.0000 | 0.0000 | 0.0000 |
| 86 | MTC20181210-1-26 | 0.0002 | 0.0000 | 0.0000 | 0.0000 |
| 87 | MTC20181210-1-27 | 0.0002 | 0.0000 | 0.0000 | 0.0000 |
| 88 | MTC20181210-1-28 | 0.0004 | 0.0008 | 0.0012 | 0.0020 |
| 89 | MTC20181210-1-29 | 0.0010 | 0.0001 | 0.0000 | 0.0001 |
| 90 | MTC20181210-2-1 | 0.0022 | 0.0025 | 0.0048 | 0.0073 |
| 91 | MTC20181210-2-2 | 0.2988 | 0.3419 | 0.5409 | 0.8829 |
| 92 | MTC20181210-2-3 | 1.0348 | 1.3703 | 2.5379 | 3.9082 |
| 93 | MTC20181210-2-4 | 1.6090 | 2.4450 | 4.2687 | 6.7137 |
| 94 | MTC20181210-2-5 | 1.2016 | 2.1997 | 3.5974 | 5.7971 |
| 95 | MTC20181210-2-6 | 0.7964 | 1.5966 | 2.5627 | 4.1592 |
| 96 | MTC20181210-2-7 | 0.3922 | 0.9174 | 0.7681 | 1.6855 |
| 97 | MTC20181210-2-8 | 0.2379 | 0.5952 | 0.9010 | 1.4962 |
| 98 | MTC20181210-2-9 | 0.1601 | 0.4119 | 0.6344 | 1.0463 |
| 99 | MTC20181210-2-10 | 0.0858 | 0.2335 | 0.3654 | 0.5989 |
| 100 | MTC20181210-2-11 | 0.0437 | 0.1266 | 0.1981 | 0.3247 |
| 101 | MTC20181210-2-13 | 0.0174 | 0.0487 | 0.0725 | 0.1212 |
| 102 | MTC20181210-2-14 | 0.0022 | 0.0035 | 0.0021 | 0.0056 |
| 103 | MTC20181210-2-15 | 0.0015 | 0.0015 | 0.0020 | 0.0035 |
| 104 | MTC20181210-2-16 | 0.0015 | 0.0013 | 0.0014 | 0.0026 |
| 105 | MTC20181210-2-17 | 0.0006 | 0.0000 | 0.0005 | 0.0005 |
| 106 | MTC20181210-2-18 | 0.0009 | 0.0009 | 0.0000 | 0.0009 |
| 107 | MTC20181210-2-19 | 0.0015 | 0.0003 | 0.0000 | 0.0003 |
| 108 | MTC20181210-2-20 | 0.0013 | 0.0005 | 0.0000 | 0.0005 |
| 109 | MTC20181210-2-21 | 0.0008 | 0.0000 | 0.0000 | 0.0000 |
| 110 | MTC20181210-2-22 | 0.0008 | 0.0001 | 0.0000 | 0.0001 |
| 111 | MTC20181210-2-23 | 0.0004 | 0.0001 | 0.0000 | 0.0001 |
| 112 | MTC20181210-2-24 | 0.0022 | 0.0031 | 0.0043 | 0.0074 |
| 113 | MTC20181210-2-25 | 0.0023 | 0.0031 | 0.0047 | 0.0078 |
| 114 | MTC20181210-2-26 | 0.0018 | 0.0028 | 0.0043 | 0.0071 |
| 115 | MTC20181210-2-27 | 0.0016 | 0.0022 | 0.0030 | 0.0052 |
| 116 | MTC20181210-2-28 | 0.0028 | 0.0041 | 0.0058 | 0.0100 |
| 117 | MTC20181210-2-29 | 0.0008 | 0.0005 | 0.0009 | 0.0014 |
| 118 | MTC20181211-1-1 | 0.0308 | 0.0086 | 0.0323 | 0.0409 |
| 119 | MTC20181211-1-2 | 0.5042 | 0.7292 | 1.3326 | 2.0618 |
| 120 | MTC20181211-1-3 | 1.4385 | 2.0701 | 3.8081 | 5.8782 |
| 121 | MTC20181211-1-4 | 1.0700 | 1.9485 | 3.1129 | 5.0614 |
| 122 | MTC20181211-1-5 | 0.5784 | 1.2066 | 1.9594 | 3.1660 |
| 123 | MTC20181211-1-6 | 0.2924 | 0.6627 | 0.9921 | 1.6548 |
| 124 | MTC20181211-1-7 | 0.1631 | 0.4074 | 0.6154 | 1.0228 |
| 125 | MTC20181211-1-8 | 0.0577 | 0.1593 | 0.2404 | 0.3997 |
| 126 | MTC20181211-1-9 | 0.0271 | 0.0789 | 0.1187 | 0.1976 |
| 127 | MTC20181211-1-10 | 0.0124 | 0.0360 | 0.0555 | 0.0915 |
| 128 | MTC20181211-1-11 | 0.0051 | 0.0155 | 0.0227 | 0.0382 |
| 129 | MTC20181211-1-12 | 0.0045 | 0.0120 | 0.0179 | 0.0299 |
| 130 | MTC20181211-1-13 | 0.0029 | 0.0073 | 0.0104 | 0.0177 |
| 131 | MTC20181211-1-14 | 0.0012 | 0.0035 | 0.0046 | 0.0081 |
| 132 | MTC20181211-1-15 | 0.0012 | 0.0028 | 0.0037 | 0.0065 |
| 133 | MTC20181211-1-16 | 0.0007 | 0.0007 | 0.0017 | 0.0024 |
| 134 | MTC20181211-1-17 | 0.0007 | 0.0016 | 0.0004 | 0.0020 |
| 135 | MTC20181211-1-18 | 0.0015 | 0.0029 | 0.0021 | 0.0050 |
| 136 | MTC20181211-1-19 | 0.0004 | 0.0009 | 0.0007 | 0.0016 |
| 137 | MTC20181211-1-20 | 0.0004 | 0.0009 | 0.0009 | 0.0018 |
| 138 | MTC20181211-1-21 | 0.0004 | 0.0004 | 0.0002 | 0.0006 |
| 139 | MTC20181211-1-22 | 0.0003 | 0.0008 | 0.0002 | 0.0010 |
| 140 | MTC20181211-1-23 | 0.0002 | 0.0004 | 0.0000 | 0.0004 |
| 141 | MTC20181211-1-24 | 0.0004 | 0.0006 | 0.0002 | 0.0008 |
| 142 | MTC20181211-2-1 | 0.0105 | 0.0254 | 0.6093 | 0.6347 |
| 143 | MTC20181211-2-2 | 0.1149 | 0.2068 | 2.8232 | 3.0301 |
| 144 | MTC20181211-2-3 | 0.3105 | 0.5369 | 1.7742 | 2.3112 |
| 145 | MTC20181211-2-4 | 0.8629 | 1.5484 | 1.1068 | 2.6552 |
| 146 | MTC20181211-2-5 | 0.4954 | 0.9497 | 0.4676 | 1.4173 |
| 147 | MTC20181211-2-6 | 0.3439 | 0.6779 | 0.6455 | 1.3234 |
| 148 | MTC20181211-2-7 | 0.2203 | 0.4483 | 0.2783 | 0.7266 |
| 149 | MTC20181211-2-8 | 0.1175 | 0.2610 | 0.1975 | 0.4586 |
| 150 | MTC20181211-2-9 | 0.0738 | 0.1711 | 0.1336 | 0.3048 |
| 151 | MTC20181211-2-10 | 0.0408 | 0.1003 | 0.0955 | 0.1957 |
| 152 | MTC20181211-2-11 | 0.0317 | 0.0752 | 0.0581 | 0.1332 |
| 153 | MTC20181211-2-12 | 0.0188 | 0.0487 | 0.0472 | 0.0959 |
| 154 | MTC20181211-2-13 | 0.0111 | 0.0309 | 0.0352 | 0.0661 |
| 155 | MTC20181211-2-14 | 0.0135 | 0.0326 | 0.0286 | 0.0611 |
| 156 | MTC20181211-2-15 | 0.0047 | 0.0137 | 0.0223 | 0.0359 |
| 157 | MTC20181211-2-16 | 0.0028 | 0.0090 | 0.0109 | 0.0199 |
| 158 | MTC20181211-2-17 | 0.0024 | 0.0071 | 0.0088 | 0.0159 |
| 159 | MTC20181211-2-18 | 0.0013 | 0.0044 | 0.0108 | 0.0152 |
| 160 | MTC20181211-2-19 | 0.0010 | 0.0031 | 0.0038 | 0.0068 |
| 161 | MTC20181211-2-20 | 0.0010 | 0.0025 | 0.0026 | 0.0051 |
| 162 | MTC20181211-2-21 | 0.0024 | 0.0051 | 0.0028 | 0.0079 |
| 163 | MTC20181211-2-22 | 0.0008 | 0.0016 | 0.0023 | 0.0039 |
| 164 | MTC20181211-2-23 | 0.0003 | 0.0009 | 0.0031 | 0.0040 |
| 165 | MTC20181211-2-24 | 0.0002 | 0.0010 | 0.0205 | 0.0216 |
| 166 | MTC20181211-2-25 | 0.0005 | 0.0011 | 0.0065 | 0.0076 |
| 167 | MTC20181211-2-26 | 0.0005 | 0.0008 | 0.0021 | 0.0029 |
| 168 | MTC20181211-2-27 | 0.0002 | 0.0005 | 0.0026 | 0.0031 |
| 169 | MTC20181211-2-28 | 0.0003 | 0.0002 | 0.0114 | 0.0116 |
| 170 | MTC20181211-2-29 | 0.0049 | 0.0102 | 0.0087 | 0.0189 |
| 171 | MTC20181211-2-30 | 0.0003 | 0.0012 | 0.0292 | 0.0304 |

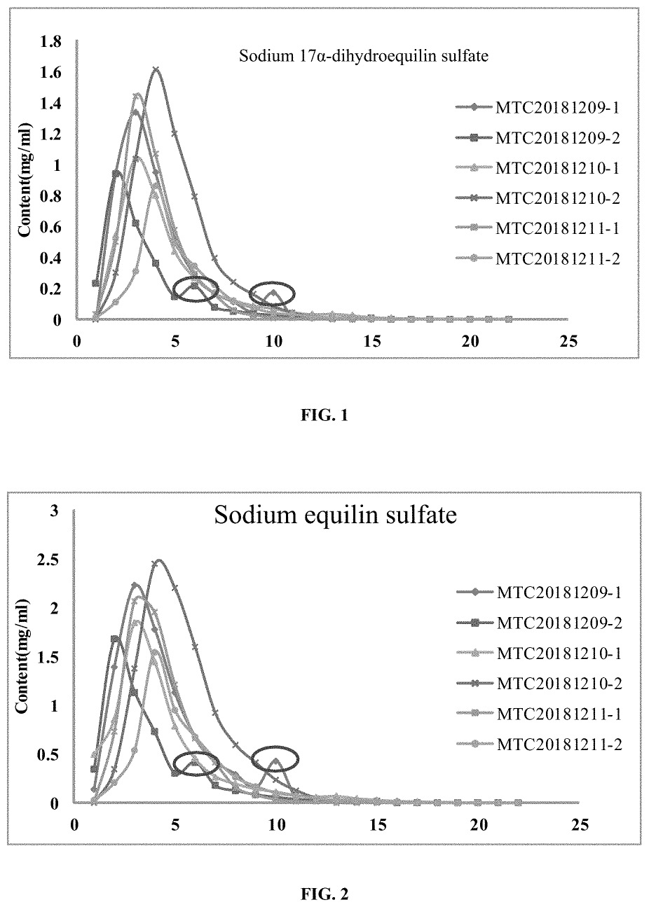

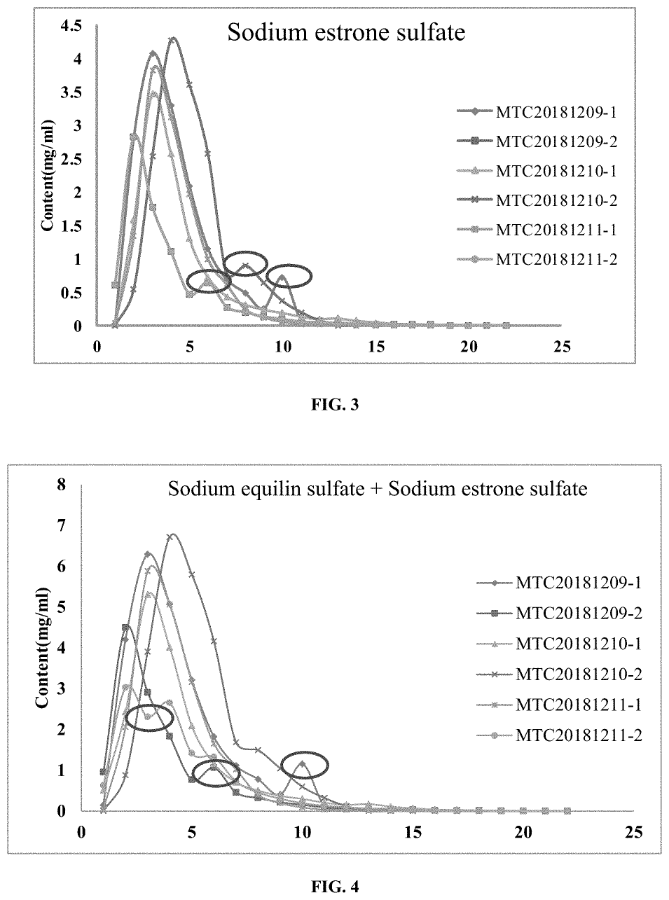

The measurement results of 171 samples obtained above were analyzed according to a trend graph for each batch, and there were abnormal measured values. As shown in FIG. 1 to FIG. 4, the abnormal content data need to be eliminated before a correction model is built.

In the present disclosure, the PMU eluate samples were subjected to NIRS with Focused Photonics NIR1500, the Mahalanobis distance method based on L1-PCA was used to eliminate abnormal sample spectral values, and spectral data of the PMU eluate samples were acquired; off-line detection was conducted under the following conditions: background: air; transmission measurement mode; wavelength detection range: 10,000 cm⁻¹ to 4,000 cm⁻¹; the number of scans: 64; resolution: 8 cm⁻¹; OPL: 2 mm; 4 repetitive scans for each PMU sample, with each measurement for 4 s on average; and spectral data: average value; and the acquired spectral data were pre-processed with the convolution-based smoothing and then subjected to band selection with the iterative optimization wavelength selection method 1, and spectral data of a characteristic band and a corresponding actual CE content value in the PMU eluate sample were subjected to regression fit by PLS to build a correction model. Specifically:

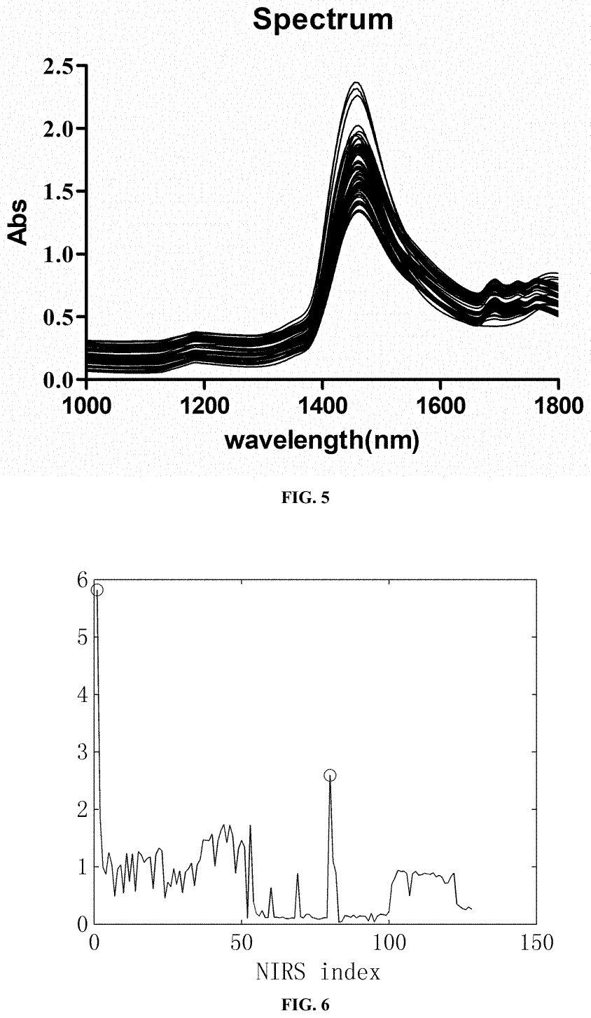

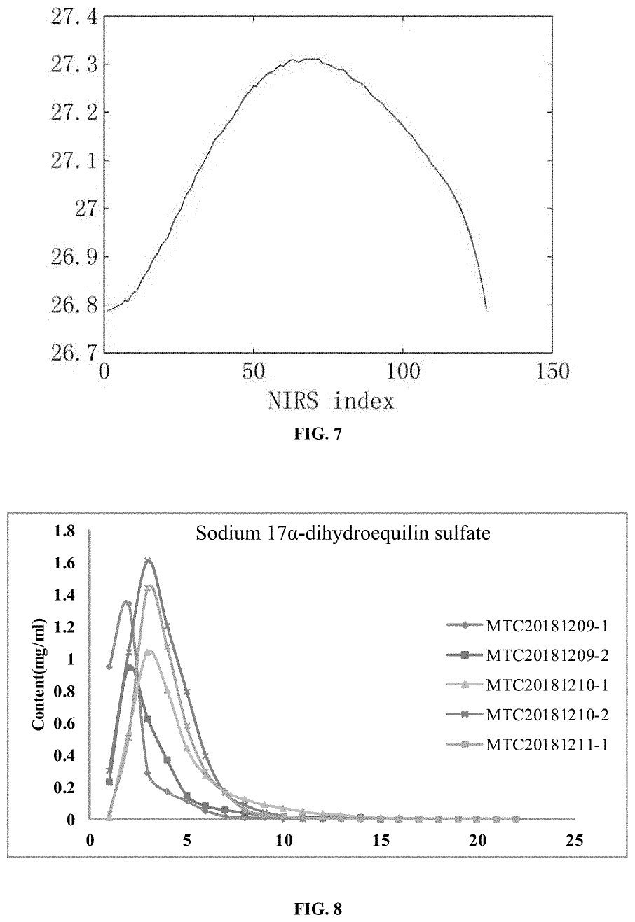

NIR spectra were acquired for the PMU eluate samples by the Focused Photonics NIR1500, and results were shown in FIG. 5. It can be seen from FIG. 5 that there are abnormal spectra. With a threshold set to 2 to 3, the abnormal spectra of MTC-20181209-1-1 and MTC-20181210-2-1 could be found out by the Mahalanobis distance method based on L1-PCA and eliminated, and then a correction model was built, as shown in FIG. 6. Abnormal spectrum calculation results of the Mahalanobis distance method were taken as a comparative example, as shown in FIG. 7. It can be seen from the comparison of FIG. 6 with FIG. 7 that the Mahalanobis distance method cannot identify abnormal spectral data, but the Mahalanobis distance method based on L1-PCA can accurately identify the abnormal spectral data, which is beneficial to improve the accuracy of detection results.

(1) During a modeling process of sodium 17α-dihydroequilin sulfate, 5 batches of collected CE samples, namely, MTC20181209-1, MTC20181209-2, MTC20181210-1, MTC20181210-2, and MTC20181211-1, were adopted as a correction set; 1 batch of CE samples, namely, MTC20181211-2, was adopted as a verification set; and with a threshold set to 2 to 3, the Mahalanobis distance method based on L1-PCA was used to eliminate abnormal spectra, and then a correction model was built and prediction was conducted for unknown samples, as shown in Table 3.

TABLE 3
Samples in the correction set for
sodium 17α-dihydroequilin sulfate

| Serial | | Sodium 17α-dihydroequilin |
|---|---|---|
| No. | Spectrum No. | sulfate (mg/mL) |
| 1 | MTC20181209-1-2 | 0.9462 |
| 2 | MTC20181209-1-3 | 1.3380 |
| 3 | MTC20181209-1-6 | 0.2888 |
| 4 | MTC20181209-1-7 | 0.1691 |
| 5 | MTC20181209-1-8 | 0.1122 |
| 6 | MTC20181209-1-9 | 0.0513 |
| 7 | MTC20181209-1-11 | 0.0152 |
| 8 | MTC20181209-1-12 | 0.0098 |
| 9 | MTC20181209-1-13 | 0.0054 |
| 10 | MTC20181209-1-14 | 0.0039 |
| 11 | MTC20181209-1-15 | 0.0028 |
| 12 | MTC20181209-1-16 | 0.0027 |
| 13 | MTC20181209-1-17 | 0.0025 |
| 14 | MTC20181209-1-18 | 0.0017 |
| 15 | MTC20181209-1-19 | 0.0018 |
| 16 | MTC20181209-1-20 | 0.0016 |
| 17 | MTC20181209-1-21 | 0.0011 |
| 18 | MTC20181209-1-22 | 0.0011 |
| 19 | MTC20181209-1-23 | 0.0007 |
| 20 | MTC20181209-1-24 | 0.0009 |
| 21 | MTC20181209-1-25 | 0.0009 |
| 22 | MTC20181209-1-26 | 0.0006 |
| 23 | MTC20181209-1-27 | 0.0005 |
| 24 | MTC20181209-1-28 | 0.0005 |
| 25 | MTC20181209-1-29 | 0.0009 |
| 26 | MTC20181209-2-1 | 0.2301 |
| 27 | MTC20181209-2-2 | 0.9438 |
| 28 | MTC20181209-2-3 | 0.6221 |
| 29 | MTC20181209-2-4 | 0.3657 |
| 30 | MTC20181209-2-5 | 0.1479 |
| 31 | MTC20181209-2-7 | 0.0803 |
| 32 | MTC20181209-2-8 | 0.0553 |
| 33 | MTC20181209-2-9 | 0.0368 |
| 34 | MTC20181209-2-10 | 0.0257 |
| 35 | MTC20181209-2-11 | 0.0147 |
| 36 | MTC20181209-2-12 | 0.0128 |
| 37 | MTC20181209-2-13 | 0.0095 |
| 38 | MTC20181209-2-14 | 0.0072 |
| 39 | MTC20181209-2-15 | 0.0058 |
| 40 | MTC20181209-2-16 | 0.0025 |
| 41 | MTC20181209-2-17 | 0.0025 |
| 42 | MTC20181209-2-18 | 0.0029 |
| 43 | MTC20181209-2-19 | 0.0009 |
| 44 | MTC20181209-2-20 | 0.0009 |
| 45 | MTC20181209-2-21 | 0.0010 |
| 46 | MTC20181209-2-22 | 0.0006 |
| 47 | MTC20181209-2-23 | 0.0009 |
| 48 | MTC20181209-2-24 | 0.0068 |
| 49 | MTC20181209-2-25 | 0.0018 |
| 50 | MTC20181209-2-26 | 0.0004 |
| 51 | MTC20181209-2-27 | 0.0011 |
| 52 | MTC20181209-2-28 | 0.0033 |
| 53 | MTC20181209-2-29 | 0.0023 |
| 54 | MTC20181209-2-30 | 0.0092 |
| 55 | MTC20181210-1-1 | 0.0061 |
| 56 | MTC20181210-1-2 | 0.5423 |
| 57 | MTC20181210-1-3 | 1.0400 |
| 58 | MTC20181210-1-4 | 0.7997 |
| 59 | MTC20181210-1-5 | 0.4399 |
| 60 | MTC20181210-1-6 | 0.2678 |
| 61 | MTC20181210-1-7 | 0.1685 |
| 62 | MTC20181210-1-8 | 0.1207 |
| 63 | MTC20181210-1-9 | 0.0882 |
| 64 | MTC20181210-1-10 | 0.0680 |
| 65 | MTC20181210-1-11 | 0.0448 |
| 66 | MTC20181210-1-12 | 0.0309 |
| 67 | MTC20181210-1-14 | 0.0245 |
| 68 | MTC20181210-1-15 | 0.0127 |
| 69 | MTC20181210-1-16 | 0.0063 |
| 70 | MTC20181210-1-17 | 0.0041 |
| 71 | MTC20181210-1-18 | 0.0025 |
| 72 | MTC20181210-1-19 | 0.0027 |
| 73 | MTC20181210-1-20 | 0.0015 |
| 74 | MTC20181210-1-21 | 0.0014 |
| 75 | MTC20181210-1-22 | 0.0008 |
| 76 | MTC20181210-1-23 | 0.0011 |
| 77 | MTC20181210-1-24 | 0.0001 |
| 78 | MTC20181210-1-25 | 0.0001 |
| 79 | MTC20181210-1-26 | 0.0002 |
| 80 | MTC20181210-1-27 | 0.0002 |
| 81 | MTC20181210-2-2 | 0.2988 |
| 82 | MTC20181210-2-3 | 1.0348 |
| 83 | MTC20181210-2-4 | 1.6090 |
| 84 | MTC20181210-2-5 | 1.2016 |
| 85 | MTC20181210-2-6 | 0.7964 |
| 86 | MTC20181210-2-7 | 0.3922 |
| 87 | MTC20181210-2-9 | 0.1601 |
| 88 | MTC20181210-2-10 | 0.0858 |
| 89 | MTC20181210-2-11 | 0.0437 |
| 90 | MTC20181210-2-13 | 0.0174 |
| 91 | MTC20181210-2-14 | 0.0022 |
| 92 | MTC20181210-2-15 | 0.0015 |
| 93 | MTC20181210-2-16 | 0.0015 |
| 94 | MTC20181210-2-17 | 0.0006 |
| 95 | MTC20181210-2-18 | 0.0009 |
| 96 | MTC20181210-2-19 | 0.0015 |
| 97 | MTC20181210-2-20 | 0.0013 |
| 98 | MTC20181210-2-21 | 0.0008 |
| 99 | MTC20181210-2-22 | 0.0008 |
| 100 | MTC20181210-2-23 | 0.0004 |
| 101 | MTC20181211-1-1 | 0.0308 |
| 102 | MTC20181211-1-2 | 0.5042 |
| 103 | MTC20181211-1-3 | 1.4385 |
| 104 | MTC20181211-1-4 | 1.0700 |
| 105 | MTC20181211-1-5 | 0.5784 |
| 106 | MTC20181211-1-6 | 0.2924 |
| 107 | MTC20181211-1-7 | 0.1631 |
| 108 | MTC20181211-1-8 | 0.0577 |
| 109 | MTC20181211-1-9 | 0.0271 |
| 110 | MTC20181211-1-10 | 0.0124 |
| 111 | MTC20181211-1-11 | 0.0051 |
| 112 | MTC20181211-1-12 | 0.0045 |
| 113 | MTC20181211-1-13 | 0.0029 |
| 114 | MTC20181211-1-14 | 0.0012 |
| 115 | MTC20181211-1-15 | 0.0012 |
| 116 | MTC20181211-1-16 | 0.0007 |
| 117 | MTC20181211-1-17 | 0.0007 |
| 118 | MTC20181211-1-18 | 0.0015 |
| 119 | MTC20181211-1-19 | 0.0004 |
| 120 | MTC20181211-1-20 | 0.0004 |
| 121 | MTC20181211-1-21 | 0.0004 |
| 122 | MTC20181211-1-22 | 0.0003 |
| 123 | MTC20181211-1-23 | 0.0002 |
| 124 | MTC20181211-1-24 | 0.0004 |

A content trend graph of the modeling sample set of sodium 17α-dihydroequilin sulfate was shown in FIG. 8.

A correction model for sodium 17α-dihydroequilin sulfate was shown in Table 4.

TABLE 4
Correction model for sodium 17α-dihydroequilin sulfate

| Pre-processing | Wavelength | Number of | | | Predicted value- | | |
|---|---|---|---|---|---|---|---|
| method for | selection | principal | | | true value | Determination | |
| spectrum | method | factors used | SECV | SEC | fitting equation | coefficient | Offset |
| Convolution- | Iterative | 10 | 0.1237 | 0.0973 | y = 0.9173 | 91.73 | 0.0012 |
| based | optimization | | | | x + 0.0128 | | |
| smoothing | wavelength | | | | | | |
| | selection | | | | | | |
| | method 1 | | | | | | |

In the predicted value-true value fitting equation in Table 4, x represents a true value and y represents a predicted value.

Prediction results of the sodium 17α-dihydroequilin sulfate samples:

The built correction model was used to predict a content in samples of batch 20181211-2, and results were shown in Table 5:

TABLE 5
Prediction results of sodium 17α-dihydroequilin
sulfate in samples of batch 20181211-2

| | | Sodium 17α-dihydroequilin sulfate (mg/mL) | | |
|---|---|---|---|---|
| Serial | | True | Predicted | Absolute |
| No. | Spectrum No. | value | value | deviation |
| 1 | MTC20181211-2-1 | 0.0105 | 0.0000 | −0.0105 |
| 2 | MTC20181211-2-2 | 0.1149 | 0.0000 | −0.1149 |
| 3 | MTC20181211-2-3 | 0.3105 | 0.6548 | 0.3443 |
| 4 | MTC20181211-2-4 | 0.8629 | 0.5171 | −0.3458 |
| 5 | MTC20181211-2-5 | 0.4954 | 0.3036 | −0.1918 |
| 6 | MTC20181211-2-6 | 0.3439 | 0.2866 | −0.0573 |
| 7 | MTC20181211-2-7 | 0.2203 | 0.0243 | −0.1960 |
| 8 | MTC20181211-2-8 | 0.1175 | 0.0000 | −0.1175 |
| 9 | MTC20181211-2-9 | 0.0738 | 0.0000 | −0.0738 |
| 10 | MTC20181211-2-10 | 0.0408 | 0.0000 | −0.0408 |
| 11 | MTC20181211-2-11 | 0.0317 | 0.0000 | −0.0317 |
| 12 | MTC20181211-2-12 | 0.0188 | 0.0000 | −0.0188 |
| 13 | MTC20181211-2-13 | 0.0111 | 0.0000 | −0.0111 |
| 14 | MTC20181211-2-14 | 0.0135 | 0.0000 | −0.0135 |
| 15 | MTC20181211-2-15 | 0.0047 | 0.0000 | −0.0047 |
| 16 | MTC20181211-2-16 | 0.0028 | 0.0000 | −0.0028 |
| 17 | MTC20181211-2-17 | 0.0024 | 0.0000 | −0.0024 |
| 18 | MTC20181211-2-18 | 0.0013 | 0.0000 | −0.0013 |
| 19 | MTC20181211-2-19 | 0.0010 | 0.0000 | −0.0010 |
| 20 | MTC20181211-2-20 | 0.0010 | 0.0000 | −0.0010 |
| 21 | MTC20181211-2-21 | 0.0024 | 0.0000 | −0.0024 |
| 22 | MTC20181211-2-22 | 0.0008 | 0.0000 | −0.0008 |
| 23 | MTC20181211-2-23 | 0.0003 | 0.0000 | −0.0003 |
| 24 | MTC20181211-2-24 | 0.0002 | 0.0000 | −0.0002 |
| 25 | MTC20181211-2-25 | 0.0005 | 0.0000 | −0.0005 |
| 26 | MTC20181211-2-26 | 0.0005 | 0.0000 | −0.0005 |
| 27 | MTC20181211-2-27 | 0.0002 | 0.0000 | −0.0002 |
| 28 | MTC20181211-2-28 | 0.0003 | 0.0000 | −0.0003 |
| 29 | MTC20181211-2-29 | 0.0049 | 0.0000 | −0.0049 |
| 30 | MTC20181211-2-30 | 0.0003 | 0.0000 | −0.0003 |

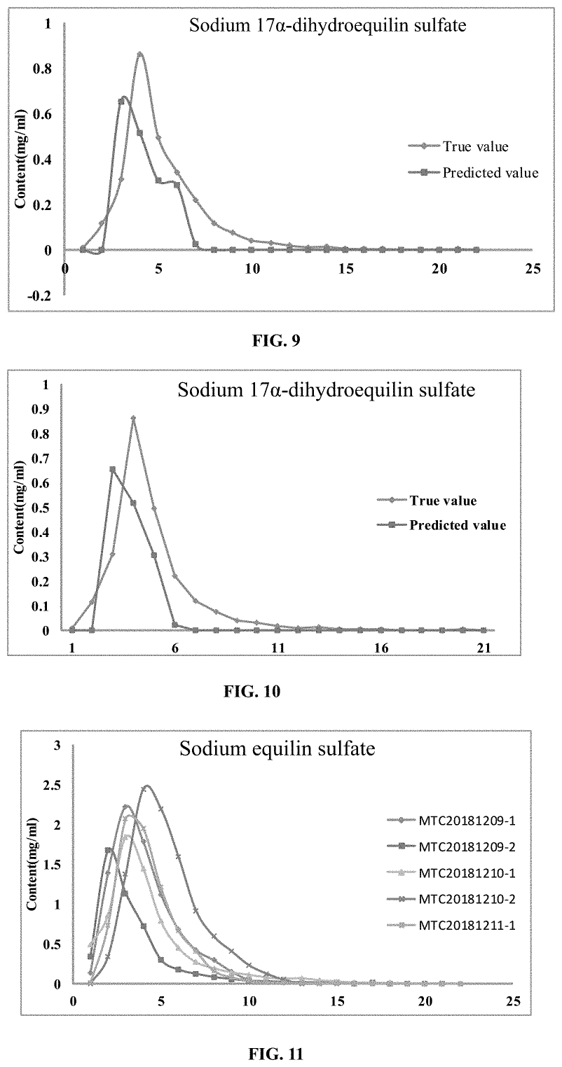

The predicted trend graph of sodium 17α-dihydroequilin sulfate in samples of batch 20181211-2 was shown in FIG. 9. It can be seen from the above verification on the correction model of sodium 17α-dihydroequilin sulfate that, in the batch 20181211-2, a predicted content trend was consistent with an actual content trend. However, during the prediction process, there was an abnormal point, namely, the 6th point during the elution process. A predicted value may show a large deviation because there occurs an error during the NIRS acquisition process or a sample is placed for too long so that a final determined content is affected. The 6th point during the elution process was eliminated, and a predicted trend graph obtained was shown in FIG. 10. It can be seen from FIG. 10 that, in the batch 20181211-2, a predicted content trend was consistent with an actual content trend.

(2) During a modeling process of sodium equilin sulfate, 5 batches of collected CE samples were adopted as a correction set; 1 batch of CE samples was adopted as a verification set; and with a threshold set to 2 to 3, the Mahalanobis distance method based on L1-PCA was used to eliminate abnormal spectra, and then a correction model was built and prediction was conducted for unknown samples, as shown in Table 6.

A content trend graph of the modeling sample set of sodium equilin sulfate was shown in FIG. 11.

A correction model for sodium equilin sulfate was shown in Table 6.

TABLE 6
Correction model for sodium equilin sulfate

| Pre-processing | Wavelength | Number of | | | Predicted value- | | |
|---|---|---|---|---|---|---|---|
| method for | selection | principal | | | true value | Determination | |
| spectrum | method | factors used | SECV | SEC | fitting equation | coefficient | Offset |
| Convolution- | Iterative | 10 | 0.2340 | 0.1722 | y = 0.9079 | 90.79 | 0.0014 |
| based | optimization | | | | x + 0.0258 | | |
| smoothing | wavelength | | | | | | |
| | selection | | | | | | |
| | method 1 | | | | | | |

In the predicted value-true value fitting equation in Table 6, x represents a true value and y represents a predicted value.

Prediction results of the sodium equilin sulfate samples:

The built correction model was used to predict a content in samples of batch 20181211-2, and results were shown in Table 7:

TABLE 7
Prediction results of sodium equilin sulfate
in samples of batch 20181211-2

| | | Sodium equilin sulfate (mg/mL) | | |
|---|---|---|---|---|
| Serial | | True | Predicted | Absolute |
| No. | Spectrum No. | value | value | deviation |
| 1 | MTC20181211-2-1 | 0.0254 | 0.0000 | −0.0254 |
| 2 | MTC20181211-2-2 | 0.2068 | 0.0000 | −0.2068 |
| 3 | MTC20181211-2-3 | 0.5369 | 1.5615 | 1.0246 |
| 4 | MTC20181211-2-4 | 1.5484 | 1.1237 | −0.4247 |
| 5 | MTC20181211-2-5 | 0.9497 | 0.0000 | −0.9497 |
| 6 | MTC20181211-2-6 | 0.6779 | 0.4602 | −0.2177 |
| 7 | MTC20181211-2-7 | 0.4483 | 0.0000 | −0.4483 |
| 8 | MTC20181211-2-8 | 0.2610 | 0.0000 | −0.2610 |
| 9 | MTC20181211-2-9 | 0.1711 | 0.0000 | −0.1711 |
| 10 | MTC20181211-2-10 | 0.1003 | 0.0000 | −0.1003 |
| 11 | MTC20181211-2-11 | 0.0752 | 0.0000 | −0.0752 |
| 12 | MTC20181211-2-12 | 0.0487 | 0.0000 | −0.0487 |
| 13 | MTC20181211-2-13 | 0.0309 | 0.0000 | −0.0309 |
| 14 | MTC20181211-2-14 | 0.0326 | 0.0000 | −0.0326 |
| 15 | MTC20181211-2-15 | 0.0137 | 0.0000 | −0.0137 |
| 16 | MTC20181211-2-16 | 0.0090 | 0.0000 | −0.0090 |
| 17 | MTC20181211-2-17 | 0.0071 | 0.0000 | −0.0071 |
| 18 | MTC20181211-2-18 | 0.0044 | 0.0000 | −0.0044 |
| 19 | MTC20181211-2-19 | 0.0031 | 0.0000 | −0.0031 |
| 20 | MTC20181211-2-20 | 0.0025 | 0.0000 | −0.0025 |
| 21 | MTC20181211-2-21 | 0.0051 | 0.0000 | −0.0051 |
| 22 | MTC20181211-2-22 | 0.0016 | 0.0000 | −0.0016 |
| 23 | MTC20181211-2-23 | 0.0009 | 0.0000 | −0.0009 |
| 24 | MTC20181211-2-24 | 0.0010 | 0.0000 | −0.0010 |
| 25 | MTC20181211-2-25 | 0.0011 | 0.2440 | 0.2429 |
| 26 | MTC20181211-2-26 | 0.0008 | 0.0000 | −0.0008 |
| 27 | MTC20181211-2-27 | 0.0005 | 0.0000 | −0.0005 |
| 28 | MTC20181211-2-28 | 0.0002 | 0.0000 | −0.0002 |
| 29 | MTC20181211-2-29 | 0.0102 | 0.0000 | −0.0102 |
| 30 | MTC20181211-2-30 | 0.0012 | 0.0000 | −0.0012 |

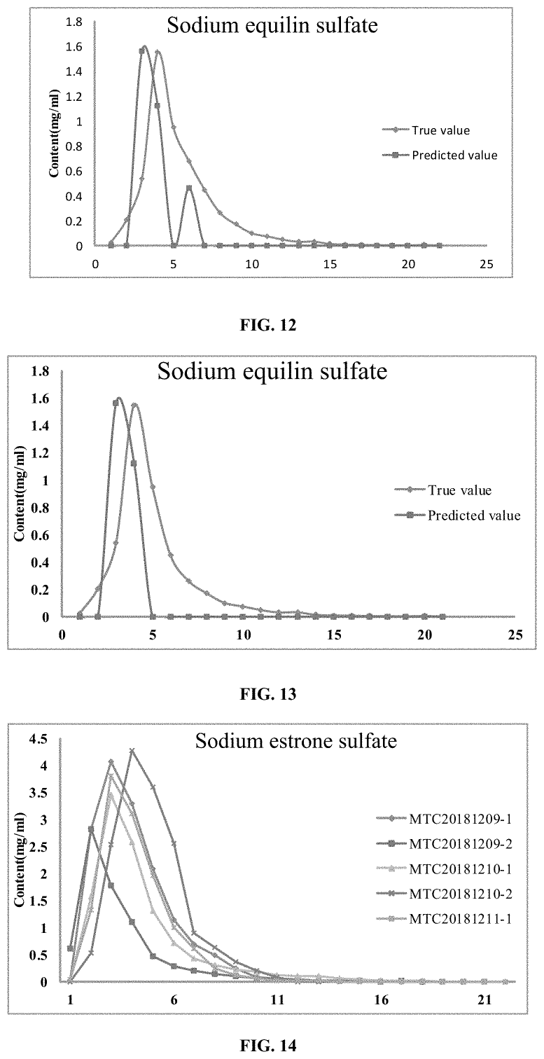

The predicted trend graph of sodium equilin sulfate in samples of batch 20181211-2 was shown in FIG. 12. It can be seen from the above verification on the correction model of sodium equilin sulfate that, in the batch 20181211-2, a predicted content trend was consistent with an actual content trend. However, during the prediction process, there were abnormal points, namely, the 6th and 25th points during the elution process. A predicted value may show a large deviation because there occurs an error during the NIRS acquisition process or a sample is placed for too long so that a final determined content is affected. The 6th and 25th points during the elution process were eliminated, and a predicted trend graph obtained was shown in FIG. 13. It can be seen from FIG. 13 that, in the batch 20181211-2, a predicted content trend was consistent with an actual content trend.

(3) During a modeling process of sodium estrone sulfate, 5 batches of collected CE samples were adopted as a correction set; 1 batch of CE samples was adopted as a verification set; and with a threshold set to 2 to 3, the Mahalanobis distance method based on L1-PCA was used to eliminate abnormal spectra, and then a model was built and prediction was conducted for unknown samples, as shown in Table 8.

A content trend graph of the modeling sample set of sodium estrone sulfate was shown in FIG. 14.

A correction model for sodium estrone sulfate was shown in Table 8.

TABLE 8
Correction model for sodium estrone sulfate

| Pre-processing | Wavelength | Number of | | | Predicted value- | | |
|---|---|---|---|---|---|---|---|
| method for | selection | principal | | | true value | Determination | |
| spectrum | method | factors used | SECV | SEC | fitting equation | coefficient | Offset |
| Convolution- | Iterative | 10 | 0.3925 | 0.2888 | y = 0.9151 | 91.51 | 0.0023 |
| based | optimization | | | | x + 0.0396 | | |
| smoothing | wavelength | | | | | | |
| | selection | | | | | | |
| | method 1 | | | | | | |

In the predicted value-true value fitting equation in Table 8, x represents a true value and y represents a predicted value.

Prediction results of the sodium estrone sulfate samples:

The built correction model was used to predict a content in samples of batch 20181211-2, and results were shown in Table 9:

TABLE 9
Prediction results of sodium estrone sulfate
in samples of batch 20181211-2

| | | Sodium estrone sulfate (mg/mL) | | |
|---|---|---|---|---|
| Serial | | True | Predicted | Absolute |
| No. | Spectrum No. | value | value | deviation |
| 1 | MTC20181211-2-1 | 0.6093 | 0.0000 | −0.6093 |
| 2 | MTC20181211-2-2 | 2.8232 | 0.0000 | −2.8232 |
| 3 | MTC20181211-2-3 | 1.7742 | 2.8469 | 1.0727 |
| 4 | MTC20181211-2-4 | 1.1068 | 1.9153 | 0.8085 |
| 5 | MTC20181211-2-5 | 0.4676 | 0.8395 | 0.3719 |
| 6 | MTC20181211-2-6 | 0.6455 | 0.7506 | 0.1051 |
| 7 | MTC20181211-2-7 | 0.2783 | 0.0000 | −0.2783 |
| 8 | MTC20181211-2-8 | 0.1975 | 0.0000 | −0.1975 |
| 9 | MTC20181211-2-9 | 0.1336 | 0.0000 | −0.1336 |
| 10 | MTC20181211-2-10 | 0.0955 | 0.0000 | −0.0955 |
| 11 | MTC20181211-2-11 | 0.0581 | 0.0000 | −0.0581 |
| 12 | MTC20181211-2-12 | 0.0472 | 0.0000 | −0.0472 |
| 13 | MTC20181211-2-13 | 0.0352 | 0.0000 | −0.0352 |
| 14 | MTC20181211-2-14 | 0.0286 | 0.0000 | −0.0286 |
| 15 | MTC20181211-2-15 | 0.0223 | 0.0000 | −0.0223 |
| 16 | MTC20181211-2-16 | 0.0109 | 0.0000 | −0.0109 |
| 17 | MTC20181211-2-17 | 0.0088 | 0.0000 | −0.0088 |
| 18 | MTC20181211-2-18 | 0.0108 | 0.0000 | −0.0108 |
| 19 | MTC20181211-2-19 | 0.0038 | 0.0000 | −0.0038 |
| 20 | MTC20181211-2-20 | 0.0026 | 0.0000 | −0.0026 |
| 21 | MTC20181211-2-21 | 0.0028 | 0.0000 | −0.0028 |
| 22 | MTC20181211-2-22 | 0.0023 | 0.0000 | −0.0023 |
| 23 | MTC20181211-2-23 | 0.0031 | 0.0000 | −0.0031 |
| 24 | MTC20181211-2-24 | 0.0205 | 0.0000 | −0.0205 |
| 25 | MTC20181211-2-25 | 0.0065 | 0.4136 | 0.4071 |
| 26 | MTC20181211-2-26 | 0.0021 | 0.0000 | −0.0021 |
| 27 | MTC20181211-2-27 | 0.0026 | 0.0000 | −0.0026 |
| 28 | MTC20181211-2-28 | 0.0114 | 0.0000 | −0.0114 |
| 29 | MTC20181211-2-29 | 0.0087 | 0.0000 | −0.0087 |
| 30 | MTC20181211-2-30 | 0.0292 | 0.0000 | −0.0292 |

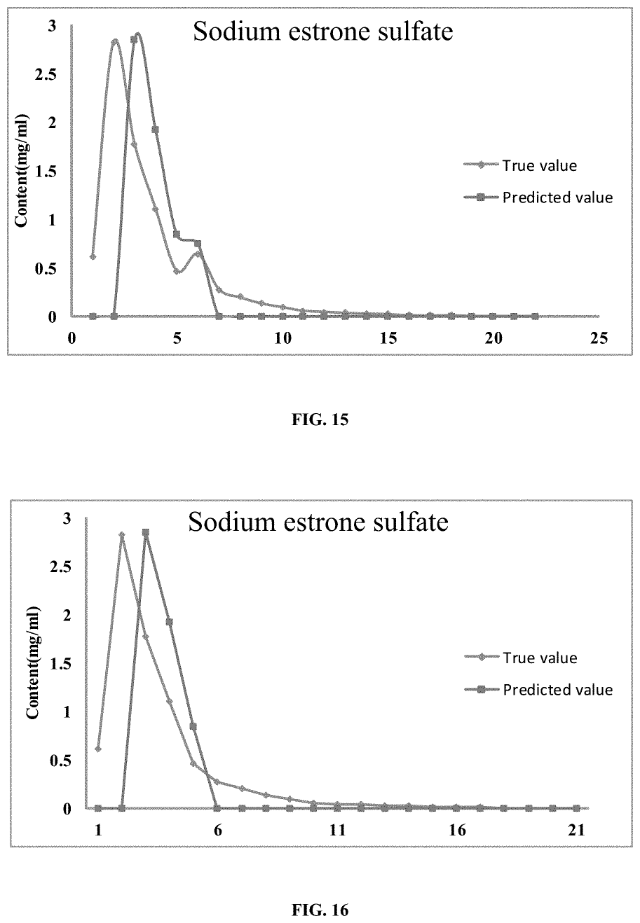

The predicted trend graph of sodium estrone sulfate in samples of batch 20181211-2 was shown in FIG. 15. It can be seen from the above verification on the correction model of sodium estrone sulfate that, in the batch 20181211-2, a predicted content trend was consistent with an actual content trend. However, during the prediction process, there were abnormal points, namely, the 6th and 25th points during the elution process. A predicted value may show a large deviation because there occurs an error during the NIRS acquisition process or a sample is placed for too long so that a final determined content is affected. The 6th and 25th points during the elution process were eliminated, and a predicted trend graph obtained was shown in FIG. 16. It can be seen from FIG. 16 that, in the batch 20181211-2, a predicted content trend was consistent with an actual content trend.

(4) During a modeling process of sodium equilin sulfate+sodium estrone sulfate, 5 batches of collected CE samples were adopted as a correction set; 1 batch of CE samples was adopted as a verification set; and with a threshold set to 2 to 3, the Mahalanobis distance method based on L1-PCA was used to eliminate abnormal spectra, and then a model was built and prediction was conducted for unknown samples, as shown in Table 10.

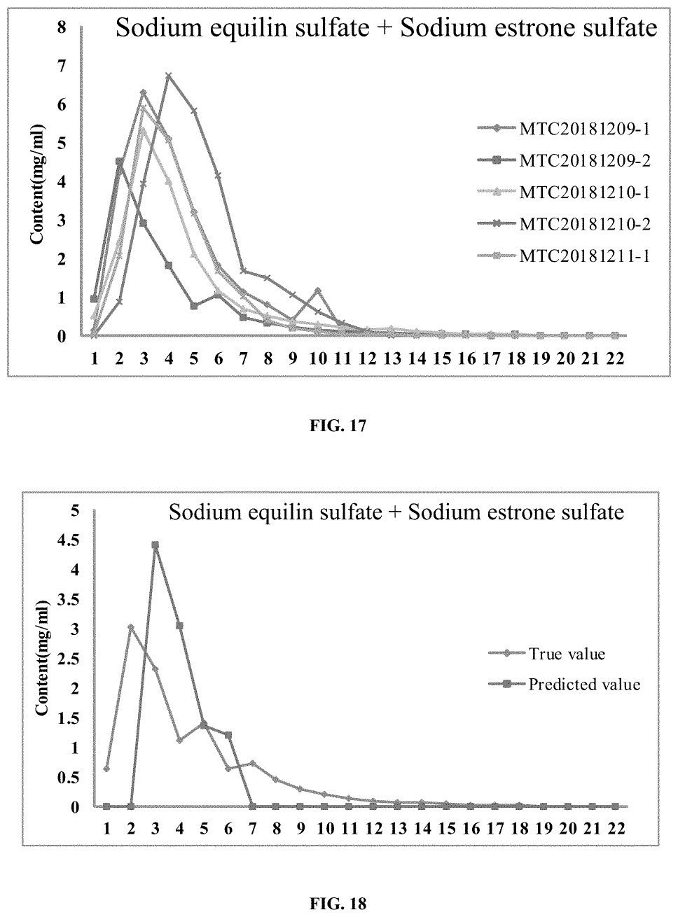

A content trend graph of the modeling sample set of sodium equilin sulfate+sodium estrone sulfate was shown in FIG. 17.

A correction model for sodium equilin sulfate+sodium estrone sulfate was shown in Table 10.

TABLE 10
Correction model for sodium equilin sulfate + sodium estrone sulfate

| Pre-processing | Wavelength | Number of | | | Predicted value- | | |
|---|---|---|---|---|---|---|---|
| method for | selection | principal | | | true value | Determination | |
| spectrum | method | factors used | SECV | SEC | fitting equation | coefficient | Offset |
| Convolution- | Iterative | 10 | 0.6192 | 0.4543 | y = 0.9148 | 91.48 | 0.0037 |
| based | optimization | | | | x + 0.0636 | | |
| smoothing | wavelength | | | | | | |
| | selection | | | | | | |
| | method 1 | | | | | | |

In the predicted value-true value fitting equation in Table 10, x represents a true value and y represents a predicted value.

Prediction results of the sodium equilin sulfate+sodium estrone sulfate samples:

The built correction model was used to predict a content in samples of batch 20181211-2, and results were shown in Table 11:

TABLE 11
Prediction results of sodium equilin sulfate +
sodium estrone sulfate in samples of batch 20181211-2

| | | Sodium equilin sulfate + | | |
|---|---|---|---|---|
| | | sodium estrone sulfate (mg/mL) | | |
| Serial | | True | Predicted | Absolute |
| No. | Spectrum No. | value | value | deviation |
| 1 | MTC20181211-2-1 | 0.6347 | 0.0000 | −0.6347 |
| 2 | MTC20181211-2-2 | 3.0301 | 0.0000 | −3.0301 |
| 3 | MTC20181211-2-3 | 2.3112 | 4.4090 | 2.0978 |
| 4 | MTC20181211-2-4 | 1.1068 | 3.0381 | 1.9313 |
| 5 | MTC20181211-2-5 | 1.4173 | 1.3682 | −0.0491 |
| 6 | MTC20181211-2-6 | 0.6455 | 1.211 | 0.5655 |
| 7 | MTC20181211-2-7 | 0.7266 | 0.0000 | −0.7266 |
| 8 | MTC20181211-2-8 | 0.4586 | 0.0000 | −0.4586 |
| 9 | MTC20181211-2-9 | 0.3048 | 0.0000 | −0.3048 |
| 10 | MTC20181211-2-10 | 0.1957 | 0.0000 | −0.1957 |
| 11 | MTC20181211-2-11 | 0.1332 | 0.0000 | −0.1332 |
| 12 | MTC20181211-2-12 | 0.0959 | 0.0000 | −0.0959 |
| 13 | MTC20181211-2-13 | 0.0661 | 0.0000 | −0.0661 |
| 14 | MTC20181211-2-14 | 0.0611 | 0.0000 | −0.0611 |
| 15 | MTC20181211-2-15 | 0.0359 | 0.0000 | −0.0359 |
| 16 | MTC20181211-2-16 | 0.0199 | 0.0000 | −0.0199 |
| 17 | MTC20181211-2-17 | 0.0159 | 0.0000 | −0.0159 |
| 18 | MTC20181211-2-18 | 0.0152 | 0.0000 | −0.0152 |
| 19 | MTC20181211-2-19 | 0.0068 | 0.0000 | −0.0068 |
| 20 | MTC20181211-2-20 | 0.0051 | 0.0000 | −0.0051 |
| 21 | MTC20181211-2-21 | 0.0079 | 0.0000 | −0.0079 |
| 22 | MTC20181211-2-22 | 0.0039 | 0.0000 | −0.0039 |
| 23 | MTC20181211-2-23 | 0.0040 | 0.0000 | −0.0040 |
| 24 | MTC20181211-2-24 | 0.0216 | 0.0000 | −0.0216 |
| 25 | MTC20181211-2-25 | 0.0076 | 0.6579 | 0.6503 |
| 26 | MTC20181211-2-26 | 0.0029 | 0.0000 | −0.0029 |
| 27 | MTC20181211-2-27 | 0.0031 | 0.0000 | −0.0031 |
| 28 | MTC20181211-2-28 | 0.0116 | 0.0000 | −0.0116 |
| 29 | MTC20181211-2-29 | 0.0189 | 0.0000 | −0.0189 |
| 30 | MTC20181211-2-30 | 0.0304 | 0.0000 | −0.0304 |

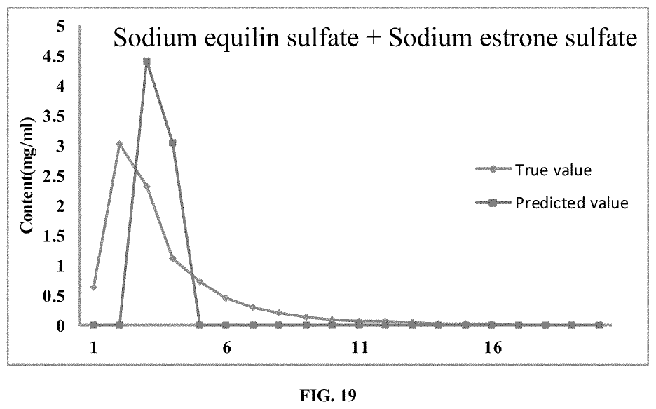

The predicted trend graph of sodium equilin sulfate+sodium estrone sulfate in samples of batch 20181211-2 was shown in FIG. 18. It can be seen from the above verification on the correction model of sodium equilin sulfate+sodium estrone sulfate that, in the batch 20181211-2, a predicted content trend was consistent with an actual content trend. However, during the prediction process, there were abnormal points, namely, the 6th and 25th points during the elution process. A predicted value may show a large deviation because there occurs an error during the NIRS acquisition process or a sample is placed for too long so that a final determined content is affected. The 5th, 6th, and 25th points during the elution process were eliminated, and a predicted trend graph obtained was shown in FIG. 19. It can be seen from FIG. 19 that, in the batch 20181211-2, a predicted content trend was consistent with an actual content trend.

### Comparative Example

The operations were basically the same as Example 1 except that abnormal spectral data were not eliminated. A model was built with abnormal spectral data being included, and results were as follows:

A correction model for sodium 17α-dihydroequilin sulfate was shown in Table 12.

TABLE 12
Correction model for sodium 17α-dihydroequilin sulfate

| Pre-processing | Wavelength | Number of | | | Predicted value- | | |
|---|---|---|---|---|---|---|---|
| method for | selection | principal | | | true value | Determination | |
| spectrum | method | factors used | SECV | SEC | fitting equation | coefficient | Offset |
| Convolution- | Iterative | 10 | 0.1486 | 0.1036 | y = 0.8851 | 88.51 | 0.003 |
| based | optimization | | | | x + 0.0146 | | |
| smoothing | wavelength | | | | | | |
| | selection | | | | | | |
| | method 1 | | | | | | |

In the predicted value-true value fitting equation in Table 12, x represents a true value and y represents a predicted value.

Prediction results of the sodium 17α-dihydroequilin sulfate samples:

The built correction model was used to predict a content in samples of batch 20181211-2, and results were shown in Table 13:

TABLE 13
Prediction results of sodium 17α-dihydroequilin
sulfate in samples of batch 20181211-2

| | | Sodium 17α-dihydroequilin sulfate (mg/mL) | | |
|---|---|---|---|---|
| Serial | | True | Predicted | Absolute |
| No. | Spectrum No. | value | value | deviation |
| 1 | MTC20181211-2-1 | 0.0105 | 0 | −0.0105 |
| 2 | MTC20181211-2-2 | 0.1149 | 0 | −0.1149 |
| 3 | MTC20181211-2-3 | 0.3105 | 0.8548 | 0.5443 |
| 4 | MTC20181211-2-4 | 0.8629 | 0.4171 | −0.4458 |
| 5 | MTC20181211-2-5 | 0.4954 | 0.7036 | 0.2082 |
| 6 | MTC20181211-2-6 | 0.3439 | 0.8626 | 0.5187 |
| 7 | MTC20181211-2-7 | 0.2203 | 0.0321 | −0.1882 |
| 8 | MTC20181211-2-8 | 0.1175 | 0 | −0.1175 |
| 9 | MTC20181211-2-9 | 0.0738 | 0 | −0.0738 |
| 10 | MTC20181211-2-10 | 0.0408 | 0 | −0.0408 |
| 11 | MTC20181211-2-11 | 0.0317 | 0 | −0.0317 |
| 12 | MTC20181211-2-12 | 0.0188 | 0 | −0.0188 |
| 13 | MTC20181211-2-13 | 0.0111 | 0 | −0.0111 |
| 14 | MTC20181211-2-14 | 0.0135 | 0 | −0.0135 |
| 15 | MTC20181211-2-15 | 0.0047 | 0 | −0.0047 |
| 16 | MTC20181211-2-16 | 0.0028 | 0 | −0.0028 |
| 17 | MTC20181211-2-17 | 0.0024 | 0 | −0.0024 |
| 18 | MTC20181211-2-18 | 0.0013 | 0 | −0.0013 |
| 19 | MTC20181211-2-19 | 0.0010 | 0 | −0.001 |
| 20 | MTC20181211-2-20 | 0.0010 | 0 | −0.001 |
| 21 | MTC20181211-2-21 | 0.0024 | 0 | −0.0024 |
| 22 | MTC20181211-2-22 | 0.0008 | 0 | −0.0008 |
| 23 | MTC20181211-2-23 | 0.0003 | 0 | −0.0003 |
| 24 | MTC20181211-2-24 | 0.0002 | 0 | −0.0002 |
| 25 | MTC20181211-2-25 | 0.0005 | 0 | −0.0005 |
| 26 | MTC20181211-2-26 | 0.0005 | 0 | −0.0005 |
| 27 | MTC20181211-2-27 | 0.0002 | 0 | −0.0002 |
| 28 | MTC20181211-2-28 | 0.0003 | 0 | −0.0003 |
| 29 | MTC20181211-2-29 | 0.0049 | 0 | −0.0049 |
| 30 | MTC20181211-2-30 | 0.0003 | 0 | −0.0003 |

A correction model for sodium equilin sulfate was shown in Table 14.

TABLE 14
Correction model for sodium equilin sulfate

| Pre-processing | Wavelength | Number of | | | Predicted value- | | |
|---|---|---|---|---|---|---|---|
| method for | selection | principal | | | true value | Determination | |
| spectrum | method | factors used | SECV | SEC | fitting equation | coefficient | Offset |
| Convolution- | Iterative | 10 | 0.2464 | 0.1946 | y = 0.8739 | 87.39 | 0.0022 |
| based | optimization | | | | x + 0.0267 | | |
| smoothing | wavelength | | | | | | |
| | selection | | | | | | |
| | method 1 | | | | | | |

In the predicted value-true value fitting equation in Table 14, x represents a true value and y represents a predicted value.

Prediction results of the sodium equilin sulfate samples:

The built correction model was used to predict a content in samples of batch 20181211-2, and results were shown in Table 15:

TABLE 15
Prediction results of sodium equilin sulfate
in samples of batch 20181211-2

| | | Sodium equilin sulfate (mg/mL) | | |
|---|---|---|---|---|
| Serial | | True | Predicted | Absolute |
| No. | Spectrum No. | value | value | deviation |
| 1 | MTC20181211-2-1 | 0.0254 | 0 | −0.0254 |
| 2 | MTC20181211-2-2 | 0.2068 | 0 | −0.2068 |
| 3 | MTC20181211-2-3 | 0.5369 | 1.7865 | 1.2496 |
| 4 | MTC20181211-2-4 | 1.5484 | 1.0123 | −0.5361 |
| 5 | MTC20181211-2-5 | 0.9497 | 0 | −0.9497 |
| 6 | MTC20181211-2-6 | 0.6779 | 0.3602 | −0.3177 |
| 7 | MTC20181211-2-7 | 0.4483 | 0 | −0.4483 |
| 8 | MTC20181211-2-8 | 0.2610 | 0 | −0.261 |
| 9 | MTC20181211-2-9 | 0.1711 | 0 | −0.1711 |
| 10 | MTC20181211-2-10 | 0.1003 | 0 | −0.1003 |
| 11 | MTC20181211-2-11 | 0.0752 | 0 | −0.0752 |
| 12 | MTC20181211-2-12 | 0.0487 | 0 | −0.0487 |
| 13 | MTC20181211-2-13 | 0.0309 | 0 | −0.0309 |
| 14 | MTC20181211-2-14 | 0.0326 | 0 | −0.0326 |
| 15 | MTC20181211-2-15 | 0.0137 | 0 | −0.0137 |
| 16 | MTC20181211-2-16 | 0.0090 | 0 | −0.009 |
| 17 | MTC20181211-2-17 | 0.0071 | 0 | −0.0071 |
| 18 | MTC20181211-2-18 | 0.0044 | 0 | −0.0044 |
| 19 | MTC20181211-2-19 | 0.0031 | 0 | −0.0031 |
| 20 | MTC20181211-2-20 | 0.0025 | 0 | −0.0025 |
| 21 | MTC20181211-2-21 | 0.0051 | 0 | −0.0051 |
| 22 | MTC20181211-2-22 | 0.0016 | 0 | −0.0016 |
| 23 | MTC20181211-2-23 | 0.0009 | 0 | −0.0009 |
| 24 | MTC20181211-2-24 | 0.0010 | 0 | −0.001 |
| 25 | MTC20181211-2-25 | 0.0011 | 0.244 | 0.2429 |
| 26 | MTC20181211-2-26 | 0.0008 | 0 | −0.0008 |
| 27 | MTC20181211-2-27 | 0.0005 | 0 | −0.0005 |
| 28 | MTC20181211-2-28 | 0.0002 | 0 | −0.0002 |
| 29 | MTC20181211-2-29 | 0.0102 | 0 | −0.0102 |
| 30 | MTC20181211-2-30 | 0.0012 | 0 | −0.0012 |

A correction model for sodium estrone sulfate was shown in Table 16.

TABLE 16
Correction model for sodium estrone sulfate

| Pre-processing | Wavelength | Number of | | | Predicted value- | | |
|---|---|---|---|---|---|---|---|
| method for | selection | principal | | | true value | Determination | |
| spectrum | method | factors used | SECV | SEC | fitting equation | coefficient | Offset |
| Convolution- | Iterative | 10 | 0.3852 | 0.3161 | y = 0.8795 | 87.95 | 0.0036 |
| based | optimization | | | | x + 0.0418 | | |
| smoothing | wavelength | | | | | | |
| | selection | | | | | | |
| | method 1 | | | | | | |

In the predicted value-true value fitting equation in Table 16, x represents a true value and y represents a predicted value.

Prediction results of the sodium estrone sulfate samples:

The built correction model was used to predict a content in samples of batch 20181211-2, and results were shown in Table 17:

TABLE 17
Prediction results of sodium estrone sulfate
in samples of batch 20181211-2

| | | Sodium estrone sulfate (mg/mL) | | |
|---|---|---|---|---|
| Serial | | True | Predicted | Absolute |
| No. | Spectrum No. | value | value | deviation |
| 1 | MTC20181211-2-1 | 0.6093 | 0 | −0.6093 |
| 2 | MTC20181211-2-2 | 2.8232 | 0 | −2.8232 |
| 3 | MTC20181211-2-3 | 1.7742 | 3.0478 | 1.2736 |
| 4 | MTC20181211-2-4 | 1.1068 | 2.2253 | 1.1185 |
| 5 | MTC20181211-2-5 | 0.4676 | 0.9583 | 0.4907 |
| 6 | MTC20181211-2-6 | 0.6455 | 1.2567 | 0.6112 |
| 7 | MTC20181211-2-7 | 0.2783 | 0 | −0.2783 |
| 8 | MTC20181211-2-8 | 0.1975 | 0 | −0.1975 |
| 9 | MTC20181211-2-9 | 0.1336 | 0 | −0.1336 |
| 10 | MTC20181211-2-10 | 0.0955 | 0 | −0.0955 |
| 11 | MTC20181211-2-11 | 0.0581 | 0 | −0.0581 |
| 12 | MTC20181211-2-12 | 0.0472 | 0 | −0.0472 |
| 13 | MTC20181211-2-13 | 0.0352 | 0 | −0.0352 |
| 14 | MTC20181211-2-14 | 0.0286 | 0 | −0.0286 |
| 15 | MTC20181211-2-15 | 0.0223 | 0 | −0.0223 |
| 16 | MTC20181211-2-16 | 0.0109 | 0 | −0.0109 |
| 17 | MTC20181211-2-17 | 0.0088 | 0 | −0.0088 |
| 18 | MTC20181211-2-18 | 0.0108 | 0 | −0.0108 |
| 19 | MTC20181211-2-19 | 0.0038 | 0 | −0.0038 |
| 20 | MTC20181211-2-20 | 0.0026 | 0 | −0.0026 |
| 21 | MTC20181211-2-21 | 0.0028 | 0 | −0.0028 |
| 22 | MTC20181211-2-22 | 0.0023 | 0 | −0.0023 |
| 23 | MTC20181211-2-23 | 0.0031 | 0 | −0.0031 |
| 24 | MTC20181211-2-24 | 0.0205 | 0 | −0.0205 |
| 25 | MTC20181211-2-25 | 0.0065 | 0.4136 | 0.4071 |
| 26 | MTC20181211-2-26 | 0.0021 | 0 | −0.0021 |
| 27 | MTC20181211-2-27 | 0.0026 | 0 | −0.0026 |
| 28 | MTC20181211-2-28 | 0.0114 | 0 | −0.0114 |
| 29 | MTC20181211-2-29 | 0.0087 | 0 | −0.0087 |
| 30 | MTC20181211-2-30 | 0.0292 | 0 | −0.0292 |

A correction model for sodium equilin sulfate+sodium estrone sulfate was shown in Table 18.

TABLE 18
Correction model for sodium equilin sulfate + sodium estrone sulfate

| Pre-processing | Wavelength | Number of | | | Predicted value- | | |
|---|---|---|---|---|---|---|---|
| method for | selection | principal | | | true value | Determination | |
| spectrum | method | factors used | SECV | SEC | fitting equation | coefficient | Offset |
| Convolution- | Iterative | 10 | 0.6332 | 0.4932 | y = 0.8800 | 88.00 | 0.0046 |
| based | optimization | | | | x + 0.0666 | | |
| smoothing | wavelength | | | | | | |
| | selection | | | | | | |
| | method 1 | | | | | | |

In the predicted value-true value fitting equation in Table 18, x represents a true value and y represents a predicted value.

Prediction results of the sodium equilin sulfate+sodium estrone sulfate samples:

The built correction model was used to predict a content in samples of batch 20181211-2, and results were shown in Table 19:

TABLE 19
Prediction results of sodium equilin sulfate +
sodium estrone sulfate in samples of batch 20181211-2

| | | Sodium equilin sulfate + | | |
|---|---|---|---|---|
| | | sodium estrone sulfate (mg/mL) | | |
| Serial | | True | Predicted | Absolute |
| No. | Spectrum No. | value | value | deviation |
| 1 | MTC20181211-2-1 | 0.6347 | 0 | −0.6347 |
| 2 | MTC20181211-2-2 | 3.0301 | 0 | −3.0301 |
| 3 | MTC20181211-2-3 | 2.3112 | 4.7579 | 2.0978 |
| 4 | MTC20181211-2-4 | 1.1068 | 3.3841 | 1.9313 |
| 5 | MTC20181211-2-5 | 1.4173 | 1.7268 | −0.0491 |
| 6 | MTC20181211-2-6 | 0.6455 | 1.5831 | 0.5655 |
| 7 | MTC20181211-2-7 | 0.7266 | 0 | −0.7266 |
| 8 | MTC20181211-2-8 | 0.4586 | 0 | −0.4586 |
| 9 | MTC20181211-2-9 | 0.3048 | 0 | −0.3048 |
| 10 | MTC20181211-2-10 | 0.1957 | 0 | −0.1957 |
| 11 | MTC20181211-2-11 | 0.1332 | 0 | −0.1332 |
| 12 | MTC20181211-2-12 | 0.0959 | 0 | −0.0959 |
| 13 | MTC20181211-2-13 | 0.0661 | 0 | −0.0661 |
| 14 | MTC20181211-2-14 | 0.0611 | 0 | −0.0611 |
| 15 | MTC20181211-2-15 | 0.0359 | 0 | −0.0359 |
| 16 | MTC20181211-2-16 | 0.0199 | 0 | −0.0199 |
| 17 | MTC20181211-2-17 | 0.0159 | 0 | −0.0159 |
| 18 | MTC20181211-2-18 | 0.0152 | 0 | −0.0152 |
| 19 | MTC20181211-2-19 | 0.0068 | 0 | −0.0068 |
| 20 | MTC20181211-2-20 | 0.0051 | 0 | −0.0051 |
| 21 | MTC20181211-2-21 | 0.0079 | 0 | −0.0079 |
| 22 | MTC20181211-2-22 | 0.0039 | 0 | −0.0039 |
| 23 | MTC20181211-2-23 | 0.0040 | 0 | −0.004 |
| 24 | MTC20181211-2-24 | 0.0216 | 0 | −0.0216 |
| 25 | MTC20181211-2-25 | 0.0076 | 0.6978 | 0.6503 |
| 26 | MTC20181211-2-26 | 0.0029 | 0 | −0.0029 |
| 27 | MTC20181211-2-27 | 0.0031 | 0 | −0.0031 |
| 28 | MTC20181211-2-28 | 0.0116 | 0 | −0.0116 |
| 29 | MTC20181211-2-29 | 0.0189 | 0 | −0.0189 |
| 30 | MTC20181211-2-30 | 0.0304 | 0 | −0.0304 |

Compared with the predicted values without abnormal spectra in Example 1, the predicted values with abnormal spectra in the comparative example showed a larger absolute deviation and thus were not accurate enough. From the contents recorded in the examples, it can be seen that the method provided in the present disclosure has high accuracy, and can quickly evaluate the quality of PMU eluates in a PMU column chromatography process.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A near-infrared (NIR) quality monitoring method used in column chromatography for extracting conjugated estrogens (CEs) from pregnant mare urine (PMU), comprising the following steps:
collecting an eluate obtained from column chromatography of a PMU stock solution as a to-be-tested sample;
subjecting the to-be-tested sample to near-infrared spectroscopy (NIRS) to obtain raw spectral data, eliminating abnormal spectral values from the raw spectral data by a Mahalanobis distance method based on L1-PCA, and importing spectral data obtained after the abnormal spectral values are eliminated into a correction model to obtain a CE content in the to-be-tested sample;
wherein, the correction model is a linear equation illustrating a relationship between true values and measured values, and the measured values refer to the NIR spectral data obtained after the abnormal spectral values are eliminated; and
the CEs comprise one or more of sodium 17α-dihydroequilin sulfate, sodium equilin sulfate, and sodium estrone sulfate.

2. The NIR quality monitoring method according to claim 1, wherein, a method for building the correction model comprises the following steps:
(1) subjecting the PMU stock solution to column chromatography to obtain a PMU eluate sample;
(2) subjecting the PMU eluate sample to liquid chromatography (LC) detection to obtain an actual CE content value in the PMU eluate sample;
(3) subjecting the PMU eluate sample in step (1) to NIRS to obtain raw sample spectral data, eliminating abnormal sample spectral values by the Mahalanobis distance method based on L1-PCA, and acquiring spectral data of the PMU eluate sample; and
(4) pre-processing the spectral data acquired in step (3), and subjecting pre-processed spectral data to band selection to obtain characteristic bands; and with partial least squares (PLS), subjecting spectral data of a characteristic band and a corresponding actual CE content value in the PMU eluate sample to regression fit to build a correction model;
wherein, steps (2) and (3) can be executed in any order.

3. The NIR quality monitoring method according to claim 1, wherein, correction models for different CEs are as follows:
a correction model for sodium 17α-dihydroequilin sulfate: y=0.9173x+0.0128;
a correction model for sodium equilin sulfate: y=0.9079x+0.0258;
a correction model for sodium estrone sulfate: y=0.9151x+0.0396; and
a correction model for sodium equilin sulfate+sodium estrone sulfate: y=0.9148x++0.0636; and
in the above correction models, x represents a true value and y represents a predicted value.

4. The NIR quality monitoring method according to claim 1, wherein, when a total content of CEs in the to-be-tested sample is greater than 0.001 mg/mL, it is determined as a starting point of the column chromatographic elution for PMU; and
when a total content of CEs in the to-be-tested sample is less than 0.001 mg/mL, it is determined as an end point of the column chromatographic elution for PMU.

5. The NIR quality monitoring method according to claim 1, wherein, the eliminating abnormal spectral values by the Mahalanobis distance method based on L1-PCA comprises:
building a spectral matrix from the raw spectral data;
according to a calculation formula shown in formula I, using an L1-PCA algorithm to solve the spectral matrix to obtain spectral principal components;
building a covariance matrix from the principal components according to a calculation formula shown in formula II;
calculating a Mahalanobis distance from the covariance matrix according to a calculation formula shown in formula III; and
setting a threshold and eliminating abnormal spectral values; wherein,
E₂(U,V)=min∥X′−UV∥_{L}_{₁}, formula I;
in formula I, X′ is an n×m spectral sample matrix, with n as the number of samples and m as the number of data points acquired for each spectrum; U is a projection matrix; V is a coefficient matrix; and L₁ is matrix norm 1;
S=T′T/n, formula II;
in formula II, T′ is the transposition of T, n is the number of samples, and a calculation method of T comprises: after a signal subspace P of spectral data is obtained, calculating a mean spectral vector p according to the P, and subtracting the mean spectral vector μ from each sample of the P matrix;
D=\sqrt{(P−μ)^{T}S⁻¹(P−μ)}, formula III;
in formula III, P is the signal subspace of spectral data; μ is the mean spectral vector; and S is a covariance matrix of the sample signal subspace built from T;
the threshold is 2 to 3.

6. The NIR quality monitoring method according to claim 2, wherein, parameters for the LC detection in step (2) comprise:
chromatographic column: C18 chromatographic column;
chromatographic column specification: 250 mm×4.6 mm, 5 μm, 100 A;
mobile phase: phase A and phase B, wherein, the phase A is a mixed solution of a monosodium phosphate (MSP) aqueous solution, acetonitrile, and methanol in a volume ratio of 17:2:1, and the MSP aqueous solution has a concentration of 20 mmol/L and a pH of 3.5; and the phase B is a mixed solution of a disodium phosphate (DSP) aqueous solution and acetonitrile in a volume ratio of 3:7, and the DSP aqueous solution has a concentration of 10 mmol/L and a pH of 3.5;
elution procedure in the mobile phase: 0 min to 18 min, a volume fraction of phase A: reducing from 70% to 67%; 18 min to 23 min, a volume fraction of phase A: reducing from 67% to 20%; 23 min to 28 min, a volume fraction of phase A: increasing from 20% to 70%; and 28 min to 35 min, a volume fraction of phase A: stabilizing at 70%;
flow rate: 1.0 mL/min;
column temperature: 40° C.;
detection wavelength: 205 nm; and
injection volume: 1 μL.

7. The NIR quality monitoring method according to claim 1, wherein, the NIRS is conducted under the following conditions:
on-line or off-line detection; background: air; transmission measurement mode;
wavelength detection range: 10,000 cm⁻¹ to 4,000 cm⁻¹; number of scans: 32; resolution: 8 cm⁻¹; optical path length (OPL): 2 mm; 3 to 5 repetitive scans for each to-be-tested sample; and raw spectral data: average value;
or, based on the principle of raster scanning spectroscopy, light source: tungsten halogen lamp; spectral range: 1,000 nm to 1,800 nm; detector: InGaAs detector; resolution: 8 cm⁻¹; number of scans: 32; and OPL: 1 mm.

8. The NIR quality monitoring method according to claim 2, wherein, a method for the pre-processing in step (4) comprises: one of convolution-based smoothing, first order convolution-based derivation, second order convolution-based derivation, multiplicative scatter correction (MSC), standard normal variant (SNV) transformation, and normalization, or a combination of two or more thereof.

9. The NIR quality monitoring method according to claim 2, wherein, a method of the band selection in step (4) comprises full wavelength, correlation-coefficient method for wavelength interval selection, correlated component method for wavelength interval selection, iterative optimization wavelength selection method 1, or iterative optimization wavelength selection method 2.

10. The NIR quality monitoring method according to claim 2, wherein, correction models for different CEs are as follows:
a correction model for sodium 17α-dihydroequilin sulfate: y=0.9173x+0.0128;
a correction model for sodium equilin sulfate: y=0.9079x+0.0258;
a correction model for sodium estrone sulfate: y=0.9151x+0.0396; and
a correction model for sodium equilin sulfate+sodium estrone sulfate: y=0.9148x++0.0636; and
in the above correction models, x represents a true value and y represents a predicted value.

11. The NIR quality monitoring method according to claim 2, wherein, the eliminating abnormal spectral values by the Mahalanobis distance method based on L1-PCA comprises:
building a spectral matrix from the raw spectral data;
according to a calculation formula shown in formula I, using an L1-PCA algorithm to solve the spectral matrix to obtain spectral principal components;
building a covariance matrix from the principal components according to a calculation formula shown in formula II;
calculating a Mahalanobis distance from the covariance matrix according to a calculation formula shown in formula III; and
setting a threshold and eliminating abnormal spectral values; wherein,
E₂(U,V)=min∥X′−UV∥_{L}_{₁}, formula I;
in formula I, X′ is an n×m spectral sample matrix, with n as the number of samples and m as the number of data points acquired for each spectrum; U is a projection matrix; V is a coefficient matrix; and L₁ is matrix norm 1;
S=T′T/n, formula II;
in formula II, T′ is the transposition of T, n is the number of samples, and a calculation method of T comprises: after a signal subspace P of spectral data is obtained, calculating a mean spectral vector μ according to the P, and subtracting the mean spectral vector p from each sample of the P matrix;
D=\sqrt{(P−μ)^{T}S⁻¹(P−μ)}, formula III;
in formula III, P is the signal subspace of spectral data; μ is the mean spectral vector; and S is a covariance matrix of the sample signal subspace built from T;
the threshold is 2 to 3.

12. The NIR quality monitoring method according to claim 2, wherein, the NIRS is conducted under the following conditions:
on-line or off-line detection; background: air; transmission measurement mode; wavelength detection range: 10,000 cm⁻¹ to 4,000 cm⁻¹; number of scans: 32; resolution: 8 cm⁻¹; optical path length (OPL): 2 mm; 3 to 5 repetitive scans for each to-be-tested sample; and raw spectral data: average value;
or, based on the principle of raster scanning spectroscopy, light source: tungsten halogen lamp; spectral range: 1,000 nm to 1,800 nm; detector: InGaAs detector; resolution: 8 cm⁻¹; number of scans: 32; and OPL: 1 mm.
